(19) United States
(12) Patent Application Publication
(10) Pub. No.: US 2004/0198716 A1
(43) Pub. Date: Oct. 7, 2004

(54) Cysteine protease inhimbitors

(76) Inventors: Dorit Arad, Dallas, Dallas County, TX (US); Arthur P Bollon, Dallas, TX (US); David C Young, Maryland Heights, St Louis County, MO (US); Bradley W Poland, Lebanon New London County, CT (US); Andrew S Peek, Williamsburg Iowa County, IA (US); Balin Shaw, Colchester, New London County, CT (US); Jyothi Vallurupalli, Allen Collin County, TX (US)

Correspondence Address: Sidley Austin Brown & Wood, Suite 3400, 717 North Hardwood Street, Dallas, TX, 75201, US

(21) Appl. No.: 10/467,105

(22) PCT Filed: Feb. 5, 2002
(86) PCT No.: PCT/US02/03785

Related U.S. Application Data
(62) Division of application No. 60/266,412, filed on Feb. 5, 2001.
(62) Division of application No. 60/271,216, filed on Feb. 23, 2001.

(51) Int. Cl. A61K 31/55; A61K 31/215; A61K 31/35; A61K 31/16; A61K 31/15

(52) U.S. Cl. 514/212.03; 514/460; 514/529; 514/602; 514/640; 514/613

(57) ABSTRACT

Compounds having quinone and quinone analogs useful for pharmaceutical preparations have now been found which inhibit cysteine proteases, in particular, caspases and 3C-cysteine proteases. The cysteine protease inhibitors of the present invention can be identified by their mode of action in disrupting the ability of cysteine proteases and, in particular, caspases to cleave a peptide chain. These compounds are useful in inhibiting cysteine protease or cysteine protease-like proteins and for treating infections diseases or physiopathological diseases or disorders attributed to the presence of excessive or insufficient levels of cysteine proteases.

### TECHNICAL FIELD OF THE INVENTION

The present invention relates to the use of certain classes of cysteine protease inhibitors for the treatment of various diseases including infectious diseases and diseases resulting from inappropriate apoptosis.

### BACKGROUND OF THE INVENTION

Cysteine proteases are a major family of peptide-bond-cleaving hydrolases isolated from viruses, bacterial protozoa, plants, mammals and fungi, wherein the thiol group of a cysteine residue serves as a nucleophile in the catalytic process. Normal protein degradation and processing involve a variety of mechanisms which include cysteine proteases. However, a variety of physiological disorders or diseases have been attributed to the presence of excessive or insufficient levels of cysteine proteases.

One family of cysteine proteases, the caspases (i.e., cysteinyl aspartate-specific proteinases), are involved in the conserved biochemical pathway that mediates apoptosis. Apoptosis is one method by which multicellular organisms eliminate unwanted cells. Apoptosis is achieved through an endogenous mechanism of cellular suicide directed by either internal or external signals activated by the cell. Apoptotic cells are routinely recognized and then cleared by neighboring cells or macrophages before cell lysis. In normal development, apoptosis is a means for regulating cell number, facilitating morphogenesis, and eliminating harmful, abnormal or nonessential cells. Apoptosis can also occur in response to infectious diseases or irreparable cell damage.

Inappropriate apoptosis has been implicated in a number of diseases, e.g., neurodegenerative diseases such as Alzheimer's, Parkinson's and Huntington's diseases; spinal muscular atrophy; multiple sclerosis; immune-based diseases such as immunodeficiency, hypersensitivity, and autoimmune disorders; ischemic cardiovascular and neurological diseases or injury such as stroke, myocardial infarction, spinal cord injury or transplant organ damage; inflammatory diseases such as arthritis, cholangitis, colitis, encephalitis, endocerolitis, hepatitis, pancreatitis and reperfusion injury; diabetes; alopecia; and leukemias, lymphomas and other cancers. Thus, modulators of apoptosis are a potential target for therapeutics for these diseases.

Caspases reported to be involved in apoptotic cell suicide include mammalian interleukin-1β converting enzyme (ICE) and CED-3, the product of a pro-apoptotic gene in the nematode C. elegans (Ellis, et al. 1991. “Mechanisms and functions of cell death,” Annu Rev Cell Biol 7:663-698; Yuan, et al. 1993. “The C. elegans cell death gene ced-3 encodes a protein similar to mammalian interleukin-1 beta-converting enzyme,” Cell 75:641-652; Nicholson, et al. 1997. “Caspases: killer proteases,” Trends Biochem Sci 22:299-306). It has been previously reported that deletion or mutation of the gene coding for CED-3 prevented apoptotic death and that transfection of genes encoding either ICE or CED-3 into cells induced apoptosis (Yuan, et al. 1993. “The C. elegans cell death gene ced-3 encodes a protein similar to mammalian interleukin-1 beta-converting enzyme,” Cell 75:641-652; Miura, et al. 1993. “Induction of apoptosis in fibroblasts by IL-1 beta-converting enzyme, a mammalian homolog of the C. elegans cell death gene ced-3,” Cell 78:653-660; Gagliardini, et al. 1994. “Prevention of vertebrate neuronal death by the crmA gene,” Science 263:826-828). Members of the ICE/ced-3 gene family have been categorized according to known function as follows: Group I, mediators of inflammation (caspase-1, including ICE; caspase4, including ICEᵣₑₗII, TX and ICH-2; and caspase-5, including ICEᵣₑₗIII; TY; caspase-11 including Ich-3; and caspase-12); Group II, effectors of apoptosis (caspase-2, including ICH-1 and mNEDD2; caspase-3, including apopain, CPP32 and YAMA; and caspase-7, including MCH3, ICE-LAP3 and CMH-1); and Group III, activators of apoptosis (caspase-6, including MCH2; caspase-8, including MACH, FLICE and MCH5; caspase-9, including ICE-LAP6 and MCH6; caspase-10; and caspase-13 including ERICE).

Modulators of caspases have been sought as novel therapeutics. Inhibitors of caspases have been reported as useful for the treatment of diseases in which excessive apoptosis occurs, including neurodegenerative diseases such as Alzheimer, Parkinson and Huntington and cardiovascular diseases such as ischemic cardiac damage. Enhancers of caspases have been shown to be useful for the treatment of diseases in which insufficient apoptosis occurs, such as cancer, viral infections and certain autoimmune diseases. (U.S. Pat. No. 5,869,519 issued to Karanewsky et al on Feb. 9, 1999; U.S. Pat. No. 5,798,442 issued to Gallant et al on Aug. 25, 1998; U.S. Pat. No. 5,877,197 issued to Karanewsky et al on Mar. 2, 1999; U.S. Pat. No. 5,968,927 issued to Karanewsky et al on Oct. 19, 1999; U.S. Pat. No. 6,004,923 issued to Spruce et al on Dec. 21, 1999; U.S. Pat. No. 6,057,333 issued to Gunaskera et al on May 2, 2000; U.S. Pat. No. 6,153,591 issued to Cai et al on Nov. 28, 2000; International Application No. WO 00/32620 of Merck Frosst Canada & Co. published on Jun. 8, 2000; International Application No. WO 00/55114 of Cai et al published on Sep. 21, 2000; International Application No. WO 00/55127 of Merck Frosst Canada & Co. published on Sep. 21, 2000; and International Application No. WO 00/61542 of Cai et al published on Oct. 19, 2000; Lee et al. 2000. “Potent and selective nonpeptide inhibitors of caspase 3 and 7 inhibit apoptosis and maintain cell functionality,” J Biol Chem 275:16007-16014; Uhal et al. 1998. “Captopril inhibits apoptosis in human lung epithelial cells: a potential antifibrotic mechanism,” Am J Physiol 275:L1013-L1017; Graczyk, P. P. 1999. Restorative Neurology Neuroscience 14:1-23.). There is a continuing need to identify compounds having caspase-modulating properties as potential treatments for these diseases.

Cysteine proteases are also produced by various viral pathogens such as Picornaviridae (e.g., genera Enterovirus, Rhinovirus, Cardiovirus, and Aphthovirus) which have been reported as causative agents in a wide variety of diseases in humans and animals including encephalitis, meningitis, hepatitis, and myocarditis, the common cold, and foot-and-mouth disease, and in plant diseases such as the potty disease in potatoes. Picomaviruses are single-stranded positive RNA viruses that are encapsulated in a protein capsid. After inclusion into the host cell, the picornaviral RNA is translated into a 247 kDa protein that is co-and post-translationally cleaved, yielding eleven (11) mature proteins. Cysteine proteases denoted 2A and 3C, which are part of the picornaviral self-polyprotein, are responsible for these cleavages. The 2A protease cleaves co-translationally between the structural and non-structural proteins, and the 3C protease cleaves post-translationally the remaining cleavage sites except one.

Recognized as important proteins in the maturation of the picornaviral life cycle, the 3C and 2A proteases have been a prime target for extensive structural and mechanistic investigations during the last few years. Recently, their mechanism and structural features have been determined (Kreisberg et al. 1995. “Mechanistic and structural features of the picornaviral 3C protease,” In Organic Reactivity: Physical and Biological Aspects, pp. 110-122). Site-directed mutagenesis studies (Cheah et al. 1990. “Site-directed mutagenesis suggests close functional relationship between a human rhinovirus 3C cysteine protease and cellular trypsin-like serine proteases,” J Biol Chem 265:7180-7187) confirmed by X-ray studies (Matthews et al. 1994. “Structure of human rhinovirus 3C protease reveals a trypsin-like polypeptide fold, RNA-binding site, and means for cleaving precursor polyprotein,” Cell 77:761-771) led to the finding that the catalytic site of 3C is composed of the following amino acids: Cys in position 146, Glu/Asp in position 71 and His in position 40. These three amino acids in the catalytic site of the 3C enzyme constitute a hybrid between the amino acids at the catalytic site of cysteine proteases and serine proteases. The 3C protease has been shown by mutagenesis and crystallography to depend on a His/Cys diad (His40, Cys146 —rhinovirus numbering). A third conserved residue in the 3C protease, Asp 71, was initially considered analogous to Asn175 (the third member in the catalytic triad of papain), however crystallography has shown this residue to be of minor catalytic importance.

While a variety of compounds have been identified to treat viral diseases by reacting with certain 3C protease or 3C protease-like proteins, which are essential to viral replication and the activity of various proteins (e.g., Albeck et al. 1996. “Peptidyl epoxides: novel selective inactivators of cysteine proteases,” J Am Chem Soc 118:3591-3596; Ando, et al. 1993. “A new class of proteinase inhibitor. Cyclopropenone-containing inhibitor of papain,” J Am Chem Soc 115:1174-1175; Bromine et al. 1996. “Peptidyl vinyl sulphones: a new class of potent and selective cysteine protease inhibitors,” Biochem J 315:85-89; Dragovich et al. 1998. “Structure-based design, synthesis, and biological evaluation of irreversible human rhinovirus 3C protease inhibitors. 2. Peptide structure-activity studies,” J Med Chem 41:2819-2834; Kadam et al. 1994. “Citrinin hydrate and radicinin: human rhinovirus 3C-protease inhibitors discovered in a target-directed microbial screen,” J Antibiotics 47:836-839; Kong et al. 1998. “Synthesis and evaluation of peptidyl Michael acceptors that inactivate human rhinovirus 3C protease and inhibit virus replication,” J Med Chem 41:2579-2587; McCall et al. 1994. “A high capacity microbial screen for inhibitors of human rhinovirus protease 3C,” Bio/Technology 12:1012-1016; Otto, H. and Schirmeister, T. 1997. “Cysteine proteases and their inhibitors,” Chem Rev 97:133-171; Singh et al. 1991. “Structure and stereochemistry of thysanone: A novel human rhinovirus 3C-protease inhibitor from Thysanophora penicilloides,” Tetrahedron Lett 32:5279-82; Webber et al. 1996. “Design, synthesis, and evaluation of nonpeptidic inhibitors of human rhinovirus 3C protease,” J Med Chem 39:5072-82), there is a continuing need to identify antiviral compounds having 3C-protease modulating properties.

Several chemical compounds useful as inhibitors of cysteine proteases, in particular, caspases and 3C-cysteine proteases have been found. These inhibitors can be used in in vitro applications as well as pharmaceutical preparations.

### SUMMARY OF INVENTION

In one aspect, the present invention is a cysteine protease inhibitor having one of the structures represented by Formula I-CLXXVII disclosed herein. The cysteine protease inhibitors can be used to reduce apoptosis. The cysteine protease inhibitors can be used to reduce the enzymatic activity of a caspase, a caspase-3 or a 3C-protease. The cysteine protease inhibitor is used in a pharmaceutical preparation administered for treatment of a disease selected from thel group consisting of viral diseases including but not limited to picomaviruses, rhinoviruses, hepatitis viruses, immunodeficiency viruses, and influenza viruses, neurodegenerative diseases such as Alzheimer's, Parkinson's and Huntington's diseases; spinal muscular atrophy; multiple sclerosis; immune-based diseases such as immunodeficiency, hypersensitivity, and autoimmune disorders; ischemic cardiovascular and neurological diseases or injury such as stroke, myocardial infarction, spinal cord injury or transplant organ damnage; inflammatory diseases such as arthritis, cholangitis, colitis, encephalitis, endocerolitis, hepatitis, pancreatitis and reperfusion injury; diabetes; and alopecia.

In another aspect, the present invention is a method for inhibiting a cysteine protease or cysteine protease-like protein comprising contacting said cysteine protease or cysteine protease-like protein with an effective amount of a cysteine protease inhibitor having one of the structures represented by Formula I-CLXXVII disclosed herein.

In another aspect, the present invention is a method for inhibiting a cysteine protease or cysteine protease-like protein in a cell comprising contacting said cysteine protease or cysteine protease-like protein with an effective amount of a cysteine protease inhibitor having one of the structures represented by Formula I-CLXXVII disclosed herein.

In another aspect, the present invention is a method of treating a patient having a disease or disorder modulated by a cysteine protease comprising administering to said patient in need of such treatment an effective amount of a cysteine protease inhibitor having one of the structures represented by Formula I-CLXXVII disclosed herein. For treatment with a naphthoquinone, the pharmaceutical preparation preferably comprises at least one compound selected from the group consisting of DTT or a derivative, HSCH₂CH₂OHCH₂OHCH₂SH, GSH (glutathione), HOOCCH(NH₂)CH₂CH₂CONHCH(CH₂SH)CONHCH₂COOH, mycothiol (MT), any other sulfur-reducing agent, any adduct of naphthoquinone derivative and DTT or GSH or MT or any adduct with a different oxidation state (e.g., NO*⁻ or NOH*⁻), and

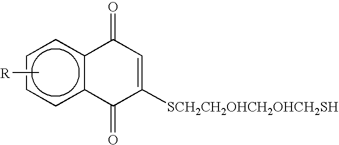

### DETAILED DESCRIPTION OF THE INVENTION

In one aspect, the present invention is a method of use of cysteine protease inhibitors presented herein for the treatment of diseases or disorders affected by cysteine protease activity. The present invention also includes pharmaceutical preparations comprising at least one of the cysteine protease inhibitors presented herein which, when administered in an effective amount, blocks the deleterious effects of infectious diseases or excessive apoptosis. The pharmaceutical preparation of the present invention can be used for the modulation of cysteine protease activity such as the picomavinis 3C-protease and 3C -protease-like proteins, i.e., proteins having similar enzyme activity and active site structure as determined by homology or by x-ray analysis. The pharmaceutical preparation of the present invention can be used for the modulation of cysteine protease activity such as caspases and caspase-like proteins, i.e., proteins having similar enzyme activity and active site structure as determined by homology or by x-ray analysis. The pharmaceutical preparation of the present invention can preferably be used for the modulation of caspase-3 and caspase-3-like proteins, i.e., proteins having similar enzyme activity and active site structure as determined by homology or by x-ray analysis.

The cysteine protease inhibitors of the present invention can be identified by their mode of action in disrupting the ability of cysteine proteases and, in particular, caspases to cleave a peptide chain. The first step in the cysteine protease catalytic mechanism is the attack of a carbonyl carbon by the cysteine sulfur atom to form a tetrahedral complex. In subsequent steps, the amide bond is broken while filling out the valence with hydrogen atoms resulting in a cleavage of the peptide bond. Inhibitors of these enzymes are compounds, which form a tetrahedral complex with the attacking sulfur but do not proceed further down the mechanistic pathway towards bond cleavage. This mode of action has been identified through biochemical and crystallographic studies. The quantitative prediction of the binding between a potential inhibitor and enzyme can be achieved using computational chemistry techniques, which provides the following criteria for cysteine protease inhibitors.

Item 1: The presence of a double bond between two dissimilar main group atoms. This ensures a location for the cysteine sulfur atom to attack, which occurs at a pi electron cloud of a sp² hybridized atom where there is a partial positive charge. Examples of this would be a carbonyl group, a thionyl group or a carbon-nitrogen double bond. Aromatic systems also have a pi electron cloud but will only have the necessary charge separation if there are two or more different elements in the conjugated system (i.e. a nitrogen atom in a conjugated ring of carbon atoms, or a Michael addition compound);

Item 2: The inhibitor will bind more strongly with the enzyme if charge separation in the pi bond being attacked by the cysteine can be increased or stabilized. This is the function of the oxyanion hole region in cysteine proteases. This can be further enhanced by the design of the molecule. For example, a carbonyl group will have a larger charge separation if there are electron withdrawing groups attached to the carbon or nearby atoms. Another way to accomplish this is by having a hydroxyl group positioned to form an intramolecular hydrogen bond with the carbonyl oxygen, as shown in a number of the preferred embodiment examples;

Item 3: The inhibitor must fit in the active site of the enzyme, to avoid steric interactions that would prevent the tetrahedral complex between enzyme and inhibitor from being formed. This is why certain compounds will not be good inhibitors, or will be inhibitors specific to one enzyme and not another; and

Item 4: The inhibitor will be more potent if the interaction between the enzyme and inhibitor via non-bond interactions such as hydrogen bonds and van der Waals interactions is strong (relative to that of other candidate inhibitors).

These criteria are generally applicable to all cysteine proteases. The cysteine protease inhibitors of the present invention are quinones and quinone analogs meeting these criteria. Functional groups for the cysteine protease inhibitors of the present invention are selected to satisfy Items 3 and 4 given above. All of the cysteine protease inhibitors of the present invention have a carbonyl group with the carbon atom being part of a ring. The geometry of the binding cleft in the caspase inhibitors of the present invention allows the insertion of these approximately planar backbones at the active site. Some of the smaller of these compounds are expected to show some inhibition of other cysteine proteases since the cysteine attack mechanism is the same. This inhibition can be quantified by use of computational chemistry techniques as a predictive tool and the use of biochemical and cell culture assays as a measurement tool.

According to the present invention, there are provided pharmaceutical preparations comprising a pharmaceutically acceptable carrier and at least one active cysteine protease inhibitor, which is a compound having the general formula given in Formula I, and the active ingredient in the pharmaceutical preparations of the present invention preferably has one of the backbone structures given Formulae II-LIX:

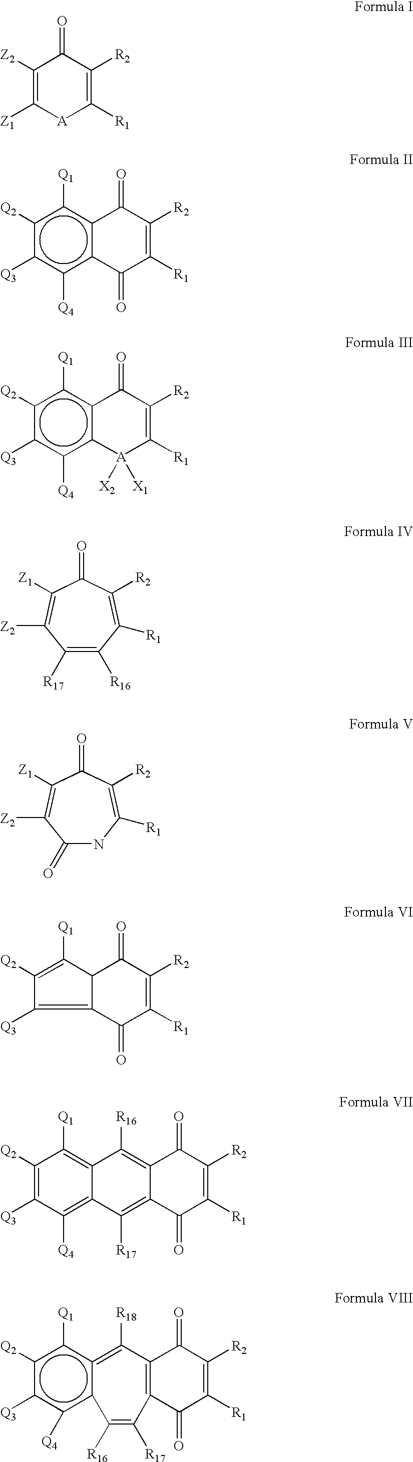

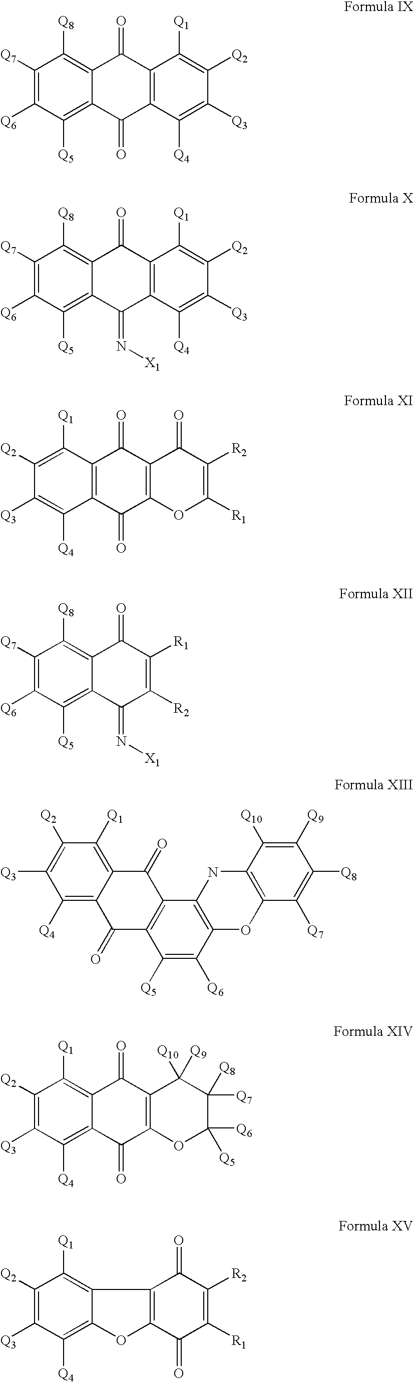

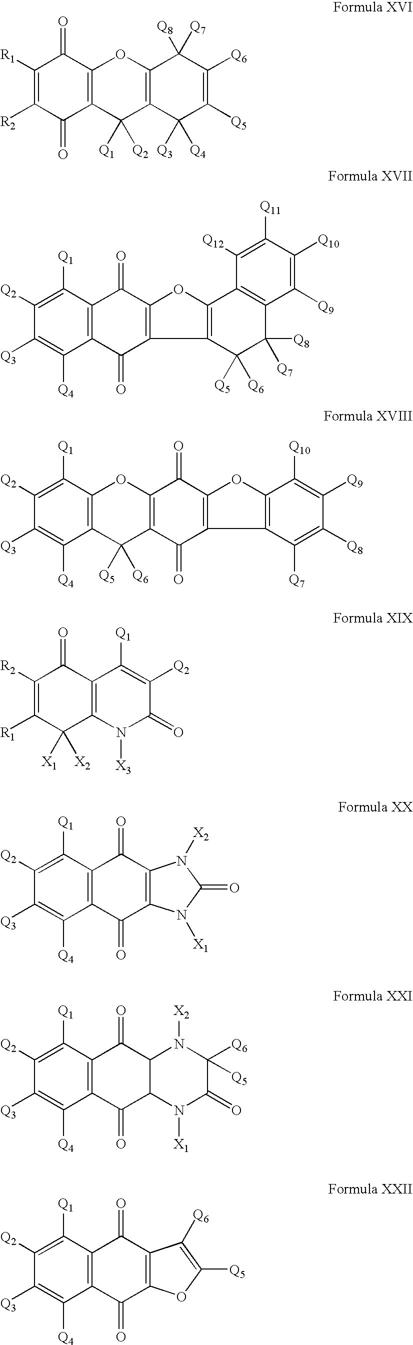

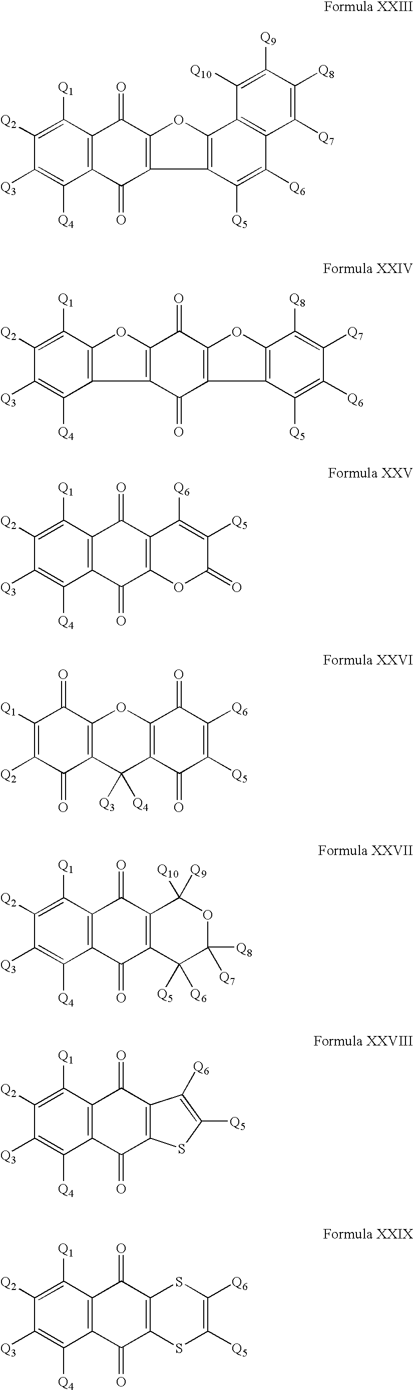

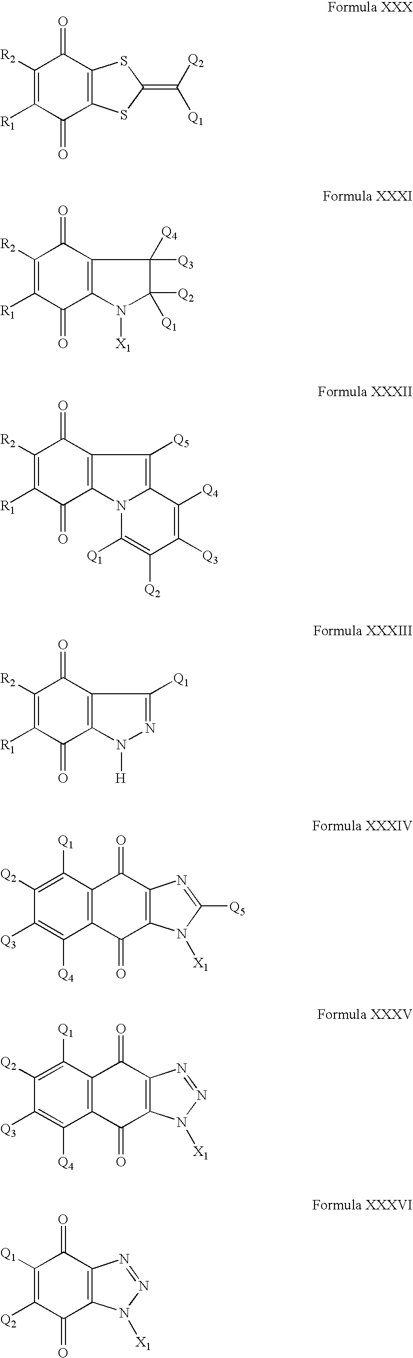

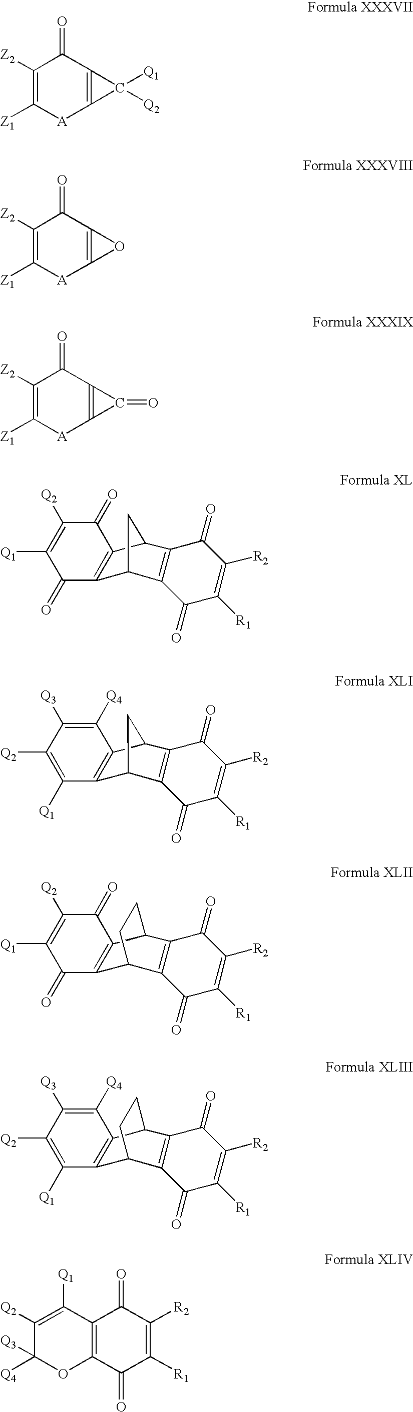

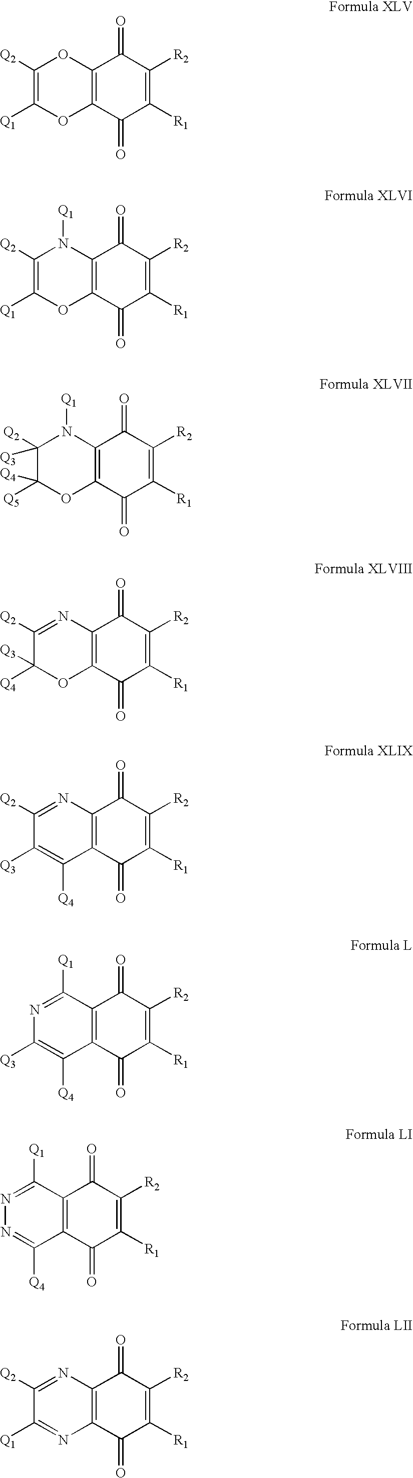

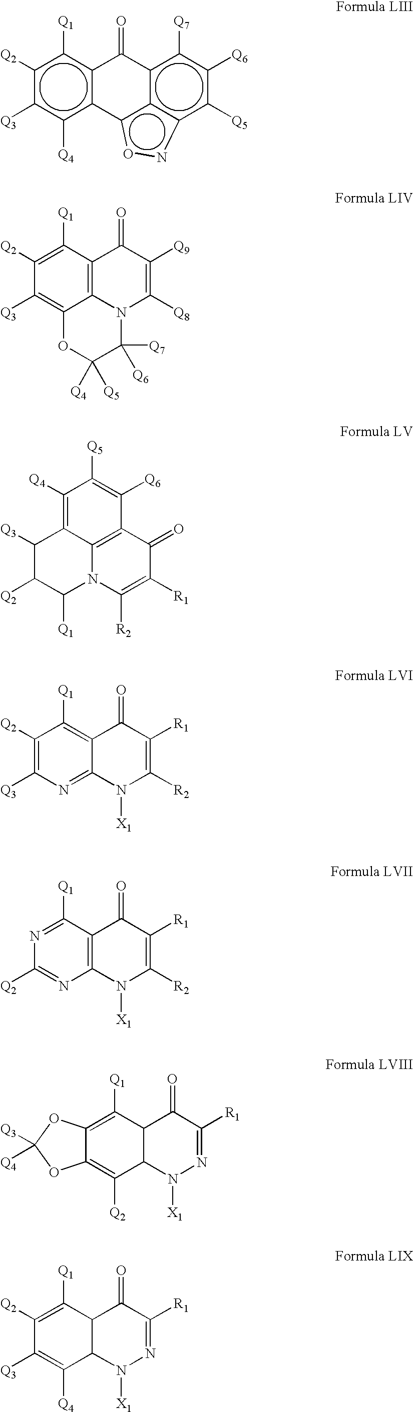

wherein

A is one of the following

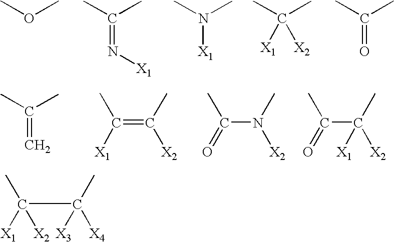

X₁, X₂, X₃, X₄ are independently hydrogen, hydroxyl, halogen, methoxy, OCH₂COOH, OCH₂CONH₂, SO₂NH₂, NHSO₂NH₂, NH-Q₁, CH₂—Q1, O—Q₁, S—Q₁, C₁-C₆ alkyl with or without substitution, C₁-C₆ alkyl ether C₁-C₆ alkyl, phenyl optionally substituted with Q₁, C₃-C₁₀ cycloalkyl or bicycloalkyl optionally substituted with Q₁, C₁-C₃ alkyloxy, —NH—CO—NH₂, —NH-(3,5-dinitro-phenyl), —NH-(2,4-dinitro-phenyl) or BCl₃;

R₁ and R₂ are independently hydrogen, hydroxyl, —COOH, 2-(5-ethyl-furan ester), 6-(2,3-dihydro-benzo[1,4]dioxine), halogen, SCH₂CH₂OH, CH₂CH₂OCH₃, morpholine, C₁-C₄ alkyl optionally substituted with R₁₀, C₂-C₄ alkenyl optionally substituted with R₁₀, or C₂-C₃ alkylyl optionally substituted with R₁₀, CF₂—R₁₀, —O-phenyl optionally substituted with R₁₀, —S-phenyl optionally substituted with R₁₀, —CH₂-phenyl optionally substituted with R₁₀, —CH₂CH═C(CH₃)₂, NH-phenyl, dimethyl amine, methyl amine, 3-hydroxy-5-oxo-tetrahydro-furan-2-yl, —NH—CH₂-phenyl optionally substituted with R₁₀, benzene sulfinyl optionally substituted with R₁₀ wherein:

R₁₀ is halogen, hydroxyl, methyl, ethyl, acetyl, carboxamide, nitro, sulfamido, phenyl or sulfamyl;

alternatively, R₁ and R₂ can form a C₃-C₁₀ cycloalkyl or bicycloalkyl, optionally containing 1 to 3 heteroatoms, optionally containing 1-3 unsaturations, and optionally substituted with hydrogen, hydroxyl, halogen, amino, nitro, cyano, C₁-C₃ alkyl optionally substituted with 1-3 R₁₁, aryl ether optionally substituted with 1-5 R₁₁, CH₂OH, CH₂SH, CF₃, CONR₁₃R₁₄, SO₂NR₁₃R₁₄, SONR₁₃R₁₄, or NR₁₅(C═O)R₁₄, wherein

R₁₁ is selected from the group consisting of halogen, cyano, nitro, amino, oxo, hydroxyl, adamantyl, carbamyl, carbamyloxy, acetyl, C₁-C₄ alkyl optionally substituted with R₁₂, C₂-C₄ alkenyl optionally substituted with R₁₂, C₂-C₃ alkylyl optionally substituted with R₁₂, C₁-C₃ aLkoxy optionally substituted with R₁₂, C₃-C₈ cycloalkyl optionally substituted with R₁₂, wherein:

R₁₂ is hydrogen, halogen, hydroxyl, methyl, ethyl, acetyl, carboxamide, nitro, sulfamido, phenyl or sulfamyl;

R₁₃ is hydrogen or hydroxyl;

R₁₄ is hydrogen, phenyl, benzyl, C₁-C₆ alkyl and C₃-C₆ cycloalkyl;

R₁₅ is hydrogen, hydroxyl, C₁-C₄ alkyl or benzyl;

Z₁ and Z₂ are hydrogen; or

alternatively Z₁ and Z₂ can form a C₁-C₅ cycloalkyl, optionally containing 1 to 3 heteroatoms, optionally contahilixg 1-3 unsaturations; and optionally substituted with hydrogen, hydroxyl, halogen, amino, nitro, cyano, C₁-C₃ alkyl optionally substituted with 1-3 R₁₁, C₁-C₃ alkoxy optionally substituted with 1-3 R₁₁, aryl ether optionally substituted with 1-5 R₁₁, CH₂OH, CH₂SH, CF₃, CONR₁₃R₁₄, SO₂NR₁₃R₁₄, SONR₁₃R₁₄, NR₁₅(C═O)R₁₄, wherein R₁₁, R₁₂, R₁₃, R₁₄, and R₁₅ are as defined above, and wherein when A is O═C—N or C═C, A can be optionally substituted with R₁₆ and R₁₇, wherein

R₁₆ and R₁₇ are hydrogen, methyl, ethyl, isopropyl, halogen, phenyl, hydroxyl, nitro, sulfamyl, or acetyl; or

alternatively Z₁ and Z₂ can form a heterocyclic ring system having a C6-C7 cycloalkyl fused to an aromatic ring, wherein the aromatic ring optionally containing 1 to 3 heteroatoms, optionally containing 1-3 unsaturations, and optionally substituted with hydrogen, hydroxyl, halogen, amino, nitro, cyano, C₁-C₃ alkyl optionally substituted with 1-3 R₁₁, C₁-C₃ aLkoxy optionally substituted with 1-3 R₁₁, aryl ether optionally substituted with 1-5 R₁₁, CH₂OH, CH₂SH, CF₃, CONR₁₃R₁₄, SO₂NR₁₃R₁₄, SONR₁₃R₁₄, or NR₁₅(C═O)R₁₄, wherein R₁₁, R₁₂, R₁₃, R₁₄, and R₁₅ are as defined above, and wherein when A is O═C—N or C═C, A can be optionally substituted with R₁₆, R₁₇, and R₁₈ wherein

R₁₆, R₁₇, and R₁₈ are hydrogen, methyl, ethyl, isopropyl, halogen, phenyl, hydroxyl, nitro, sulfamyl, or acetyl; and

Q₁-Q₁₂ are hydrogen, hydroxyl, halogen, carboxylic acid, aldehyde, phenyl, t-butyl, isopropyl, methyl, ethyl, SO₃, NH₂, CH₂—COOH, nitro, NH—CH₂—CH₂—COOH, O-cyclopropyl-NHCOCH₂CH₂COOH, CH₂-cyclopropyl-NHCOCH₂CH₂COOH, NH-cyclopropyl-NHCOCH₂CH₂COOH, OCH₂CH₂NHCOCH₂CH₂COOH, CH₂CH₂CH₂NHCOCH₂CH₂COOH, NHCH₂CH₂NHCOCH₂CH₂COOH, O-cyclopropyl-CH₂COCH₂CH₂COOH, CH₂-cyclopropyl-CH₂COCH2CH₂COOH, NH-cyclopropyl-CH₂COCH₂CH₂COOH, OCH₂CH₂CH₂COCH₂CH₂COOH, CH₂CH₂CH₂CH₂COCH₂CH₂COOH, NHCH₂CH₂CH₂COCH₂CH₂COOH, O-cyclopropyl-CH₂COCH₂CH₂Q₁, CH₂-cyclopropyl-CH₂COCH₂CH₂Q₁, NH-cyclopropyl-CH₂COCH₂CH₂Q₁, OCH₂CH₂CH₂COCH₂CH₂Q₁, CH₂CH₂CH₂CH₂COCH₂CH₂Q₁, NHCH₂CH₂CH₂COCH₂CH₂Q₁, —NHCH₂CH₂COOCH₃, —CH₂N(CH₂COOH)₂, piperazinyl, or piperadinyl.

Preferred active ingredients of the pharmaceutical preparations of the present invention are compounds selected from Formula II(i) and Formula II(ii) which have cysteine protease inhibitory activity.

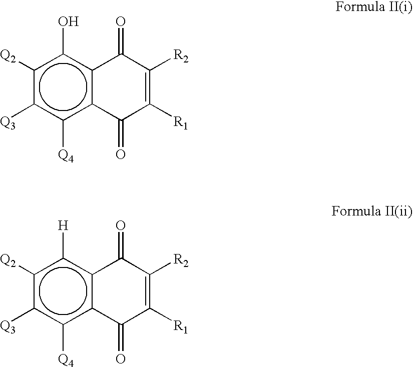

A preferred compound is Formula II(i), wherein one to four of the groups R₁, R₂, Q₂, Q₃, and Q₄ are hydrogen. Other preferred compounds include Formula II(i), wherein Q₂ and Q₄ are hydrogen, hydroxyl, halogen, C₁-C₃ alkyl optionally substituted with 1-3 R₁₁, and aryl ether optionally substituted with 1-5 R₁₁; and Formula II(ii), wherein Q₄ is hydrogen.

Preferred active ingredients of the pharmaceutical preparations of the present invention are compounds selected from Formula III(i)-Formula III(x) which have cysteine protease inhibitory activity.

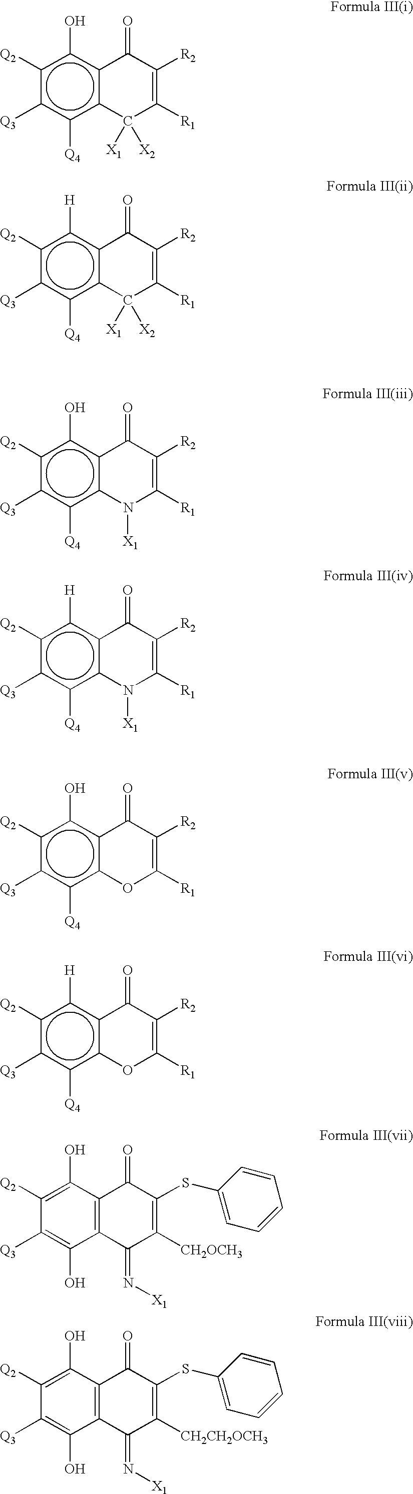

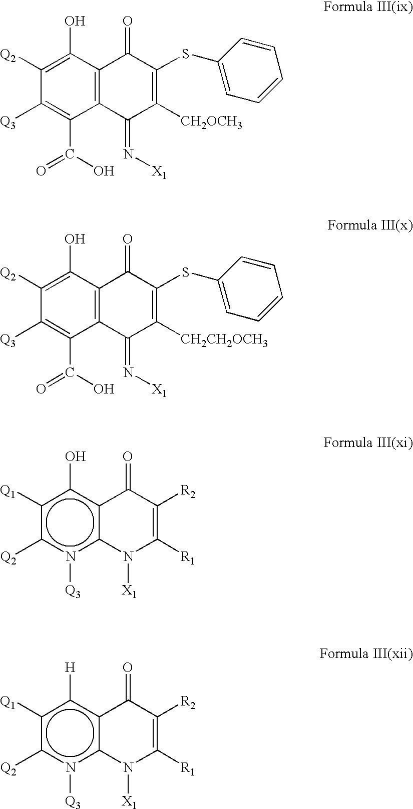

Preferred compounds include Formula III(i)-III(x), wherein Q₂ and Q₄ are hydrogen, hydroxyl, halogen, C₁-C₃ alkyl optionally substituted with 1-3 R₁₁, and aryl ether optionally substituted with 1-5 R₁₁.

Preferred active ingredients of the pharmaceutical preparations of the present invention are compounds selected from Formula IV(i) and Formula IV(ii), wherein Z₁ and Z₂ fuse to form an aromatic ring, which have cysteine protease inhibitory activity.

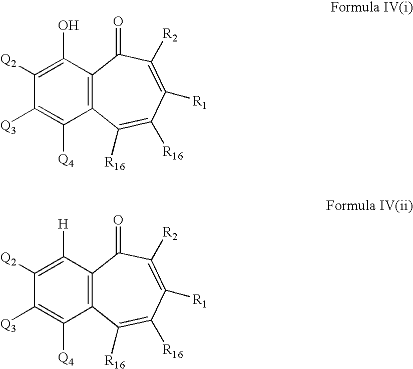

Preferred active ingredients of the pharmaceutical preparations of the present invention compounds selected from Formula V(i) and Formula V(ii), wherein Z₁ and Z₂ fuse to form an aromatic ring, which have cysteine protease inhibitory activity.

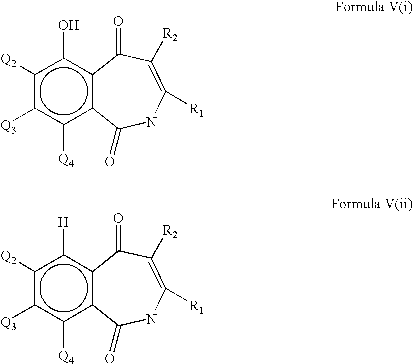

Preferred active ingredients of the pharmaceutical preparations of the present invention are compounds selected from Formula VI(i) and Formula VI(ii), wherein Z₁ and Z₂ fuse to form an indene ring, which have cysteine protease inhibitory activity.

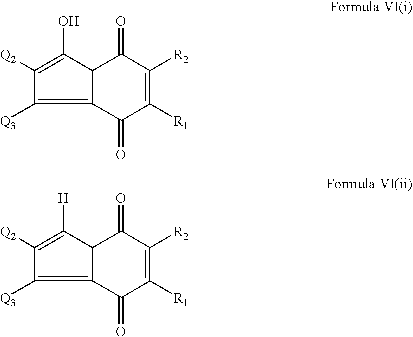

Preferred active ingredients of the pharmaceutical preparations of the present invention are compounds selected from Formula VII(i) and Formula VII(ii), wherein Z₁ and Z₂ fuse to form a heterocyclic ring system, which have cysteine protease inhibitory activity.

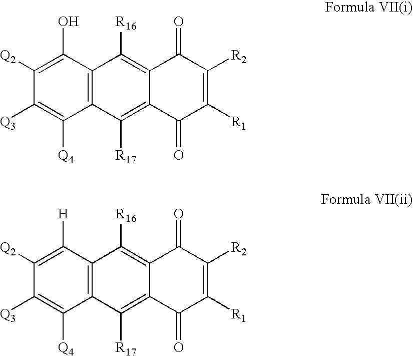

Preferred active ingredients of the pharmaceutical preparations of the present invention are compounds selected from Formula VIII(i) and Formula VIII(ii), wherein Z₁ and Z₂ fuse to form a heterocyclic ring system, which have cysteine protease inhibitory activity.

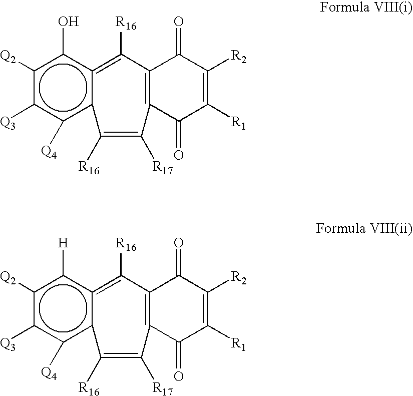

Preferred active ingredients of the pharmaceutical preparations of the present invention are compounds having Formula IX(i), wherein R₁ and R₂ fuse and Z₁ and Z₂ fuse to form a heterocyclic ring system and Q₁ is hydroxyl, which have cysteine protease inhibitory activity.

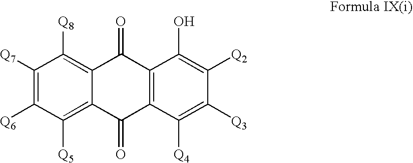

Preferred active ingredients of the pharmaceutical preparations of the present invention are compounds having Formula X(i), wherein R₁ and R₂ fuse and Z₁ and Z₂ fuse to form a heterocyclic ring system, X₁ is an unsubstituted or substituted amine, and Q₁ is hydroxyl, which have cysteine protease inhibitory activity.

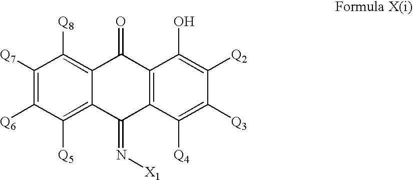

Preferred active ingredients of the pharmaceutical preparations of the present invention are compounds having Formula XI(i), wherein R₁ and R₂ fuse and Z₁ and Z₂ fuse to form a heterocyclic ring system, and Q₁ is hydroxyl, which have cysteine protease inhibitory activity.

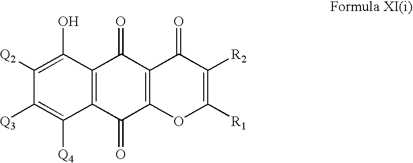

Preferred active ingredients of the pharmaceutical preparations of the present invention are compounds selected from Formula XII(i) and Formula XII(ii), wherein Z₁ and Z₂ fuse to form a heterocyclic ring system and X₁ is an unsubstituted or substituted amine, and Q₁ is hydroxyl, which have cysteine protease inhibitory activity.

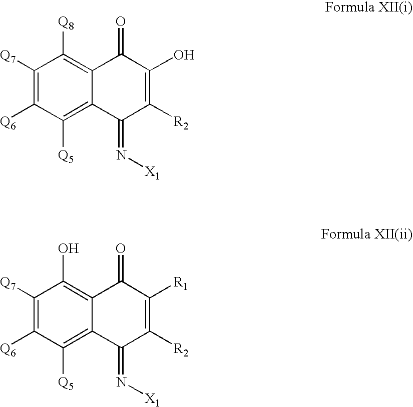

In Formula XII(i), R₁ is hydroxyl, and in Formula XII(ii), Q₈ is hydroxyl.

Preferred antiviral and 3C protease inhibitors are also based on the structure of Formula LVI.

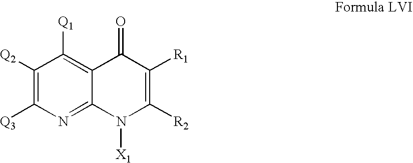

A preferred group of compounds has X₁=—CH₂CH₂CONH₂. Another preferred group of compounds has X₁=CH₂CH₃. Another preferred group is nalidixic acid and ester derivatives including C₁-C₆ alkyl, unsubstituted or substituted with an oxymethyl group, phenyl, and substituted aryl, wherein R₁, Q₂, and Q₄=H; X1=—CH₂CH₃; Q₃=—CH₃and R₂=—COOR, with R=hydroxyl, C₁-C₆ alkyl, unsubstituted or substituted with an oxymethyl group, phenyl, substituted aryl, hydrogen, methyl, ethyl, benzyl, CH₂CON(CH₂CH₃)₂, CH₂OAc, CH₂O₂CCH₂CH₃, CH₂O₂CCH₂CH₂CH₃, or CH₂O₂CCH(CH₂)₂.

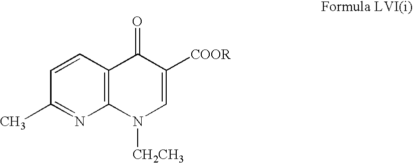

The potency of nalidixic acid as an antirhinoviral agent is 10 fold more than its potency as an antibiotic agent. These compounds can be administered as an antiviral agent against rhinoviral cold and allergic cold caused by rhinovirus, as nasal drops or nasal spray or other delivery system to the nasal mucoza, preferably the esters, which have enhanced delivery potential (Bundgaard, et al. 1989. “Enhanced delivery of nalidixic acid through human skin via acyloxymethyl ester prodrugs,” Int J Pharm 55:91-7).

One particular class of cysteine protease inhibitors disclosed herein based on Formula II(i) are naphthoquinones comprising the basic chemical structure of.

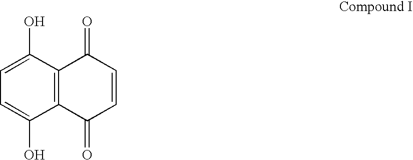

As first disclosed herein, the enantiomeric naphthoquinone natural products alkannin and shikonin, previously reported as having wound healing and antimicrobial, antithrombotic, antiamoebic, antitumor, and anti-inflammatory effects (Papageorgiou, et al. 1999. “The chemistry and biology of alkannin, shikonin, and related naphthazarin natural products,” Angew Chem Int Ed 38:270-300), have now been found to exhibit cysteine protease inhibitory activity. The chemical structure for alkannin is

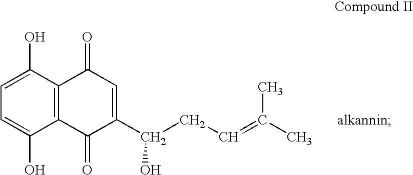

The chemical structure for shikonin is

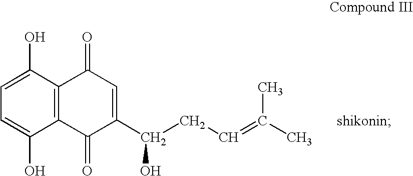

In one aspect, the present invention is a method of use of aloanin and shikonin for the treatment of diseases or disorders affected by cysteine protease activity. In another aspect, the present invention are naphthoquinone derivatives of alkannin and shikonin useful as cysteine protease inhibitors. The present invention also includes pharmaceutical preparations comprising at least one of these two compounds which, when administered in an effective amount, blocks the deleterious effects of infectious diseases or excessive apoptosis. The pharmaceutical preparations comprising alkannin and/or shikonin can be used for the modulation of cysteine protease activity such as the picornavirus 3C-protease and 3C -protease-like proteins, i.e., proteins having similar enzyme activity and active site structure as determined by homology or by x-ray analysis. The pharmaceutical preparations comprising alkannin and/or shikonin can be used for the modulation of cysteine protease activity such as caspases and caspase-like proteins, i.e., proteins having similar enzyme activity and active site structure as determined by homology or by x-ray analysis. The pharmaceutical preparations comprising alkannin and/or shikonin can preferably be used for the modulation of caspase-3 and caspase-3-like proteins, i.e., proteins having similar enzyme activity and active site structure as determined by homology or by x-ray analysis.

According to the present invention, there are provided cysteine protease inhibitors useful in pharmaceutical preparations comprising a pharmaceutically acceptable carrier and at least one active cysteine protease inhibitor, wherein the active cysteine protease inhibitor is a quinone or a derivative of quinone acting as Michael acceptor, having one of the backbone structures given below in Formulae LX-CLXXVII,

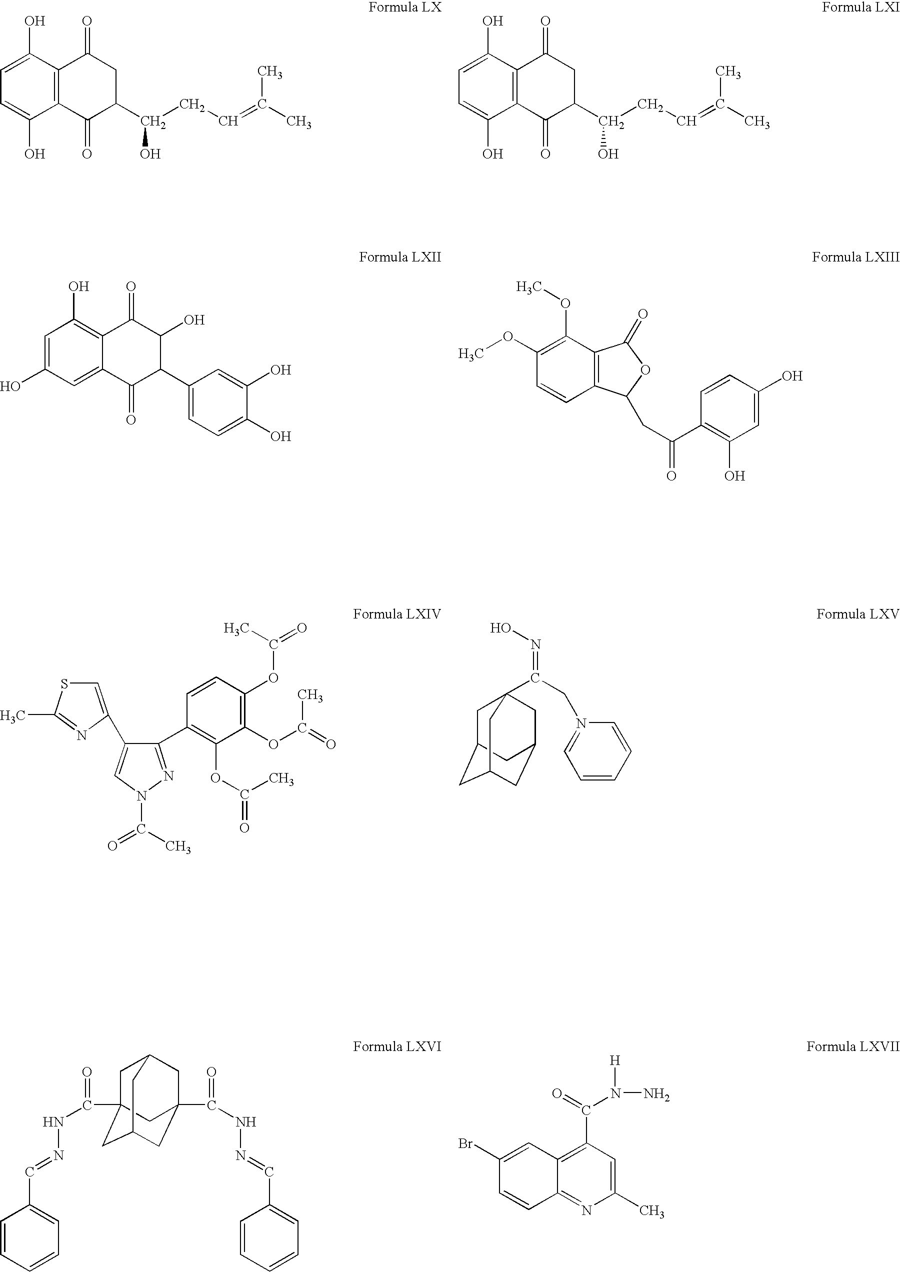

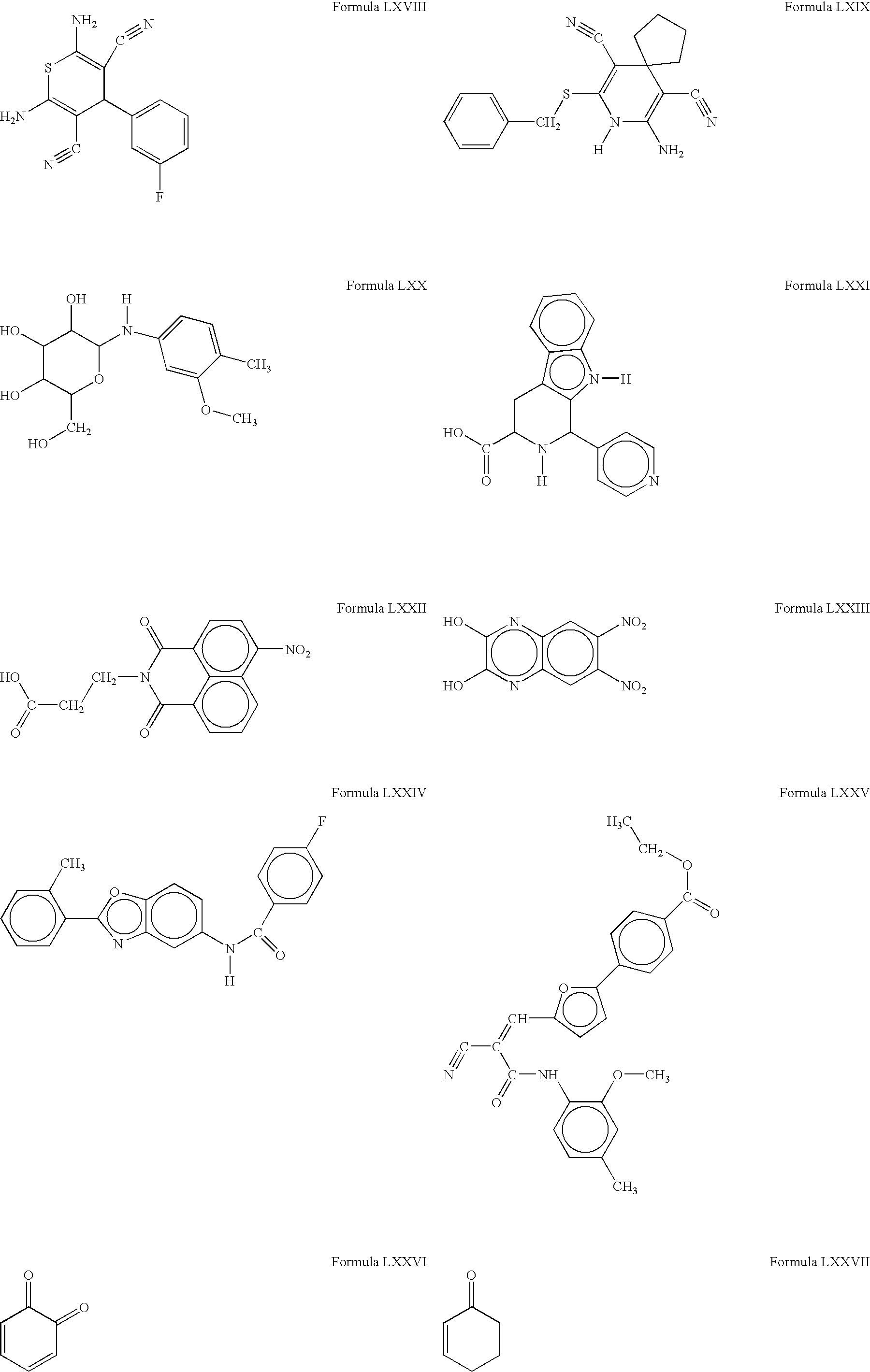

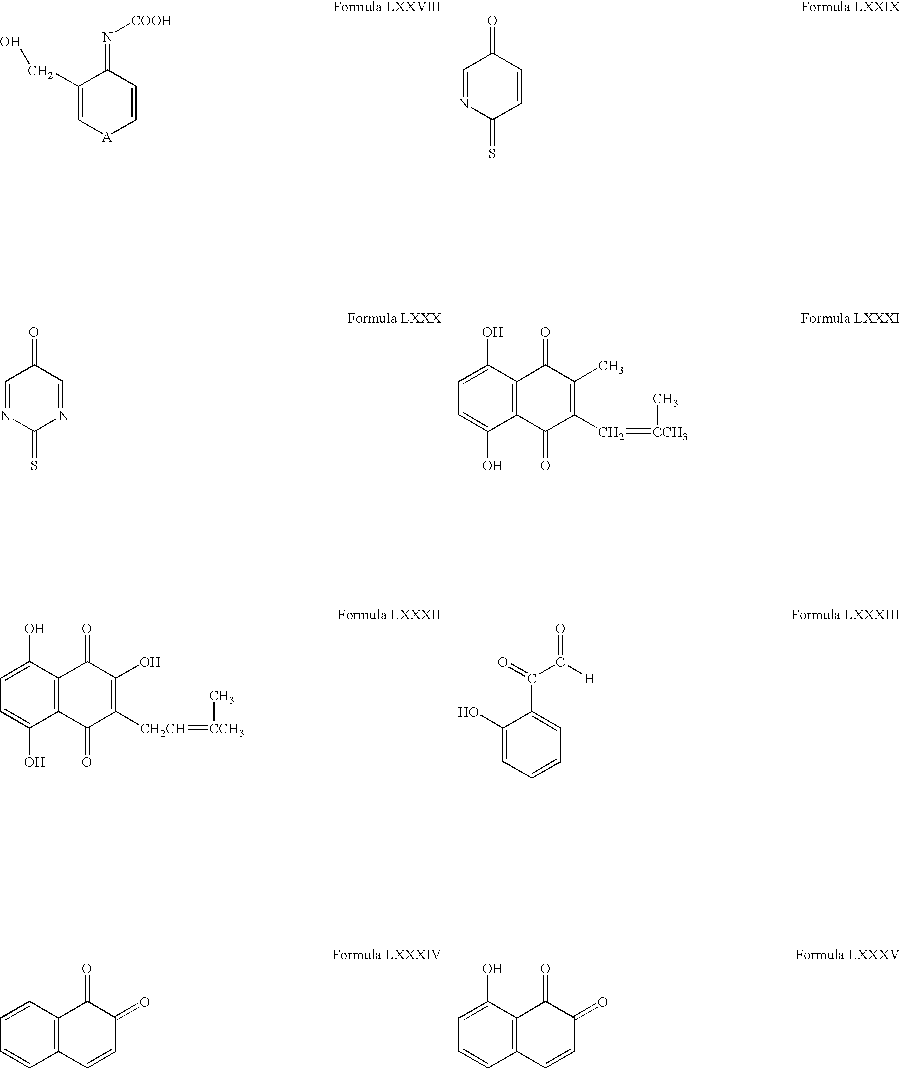

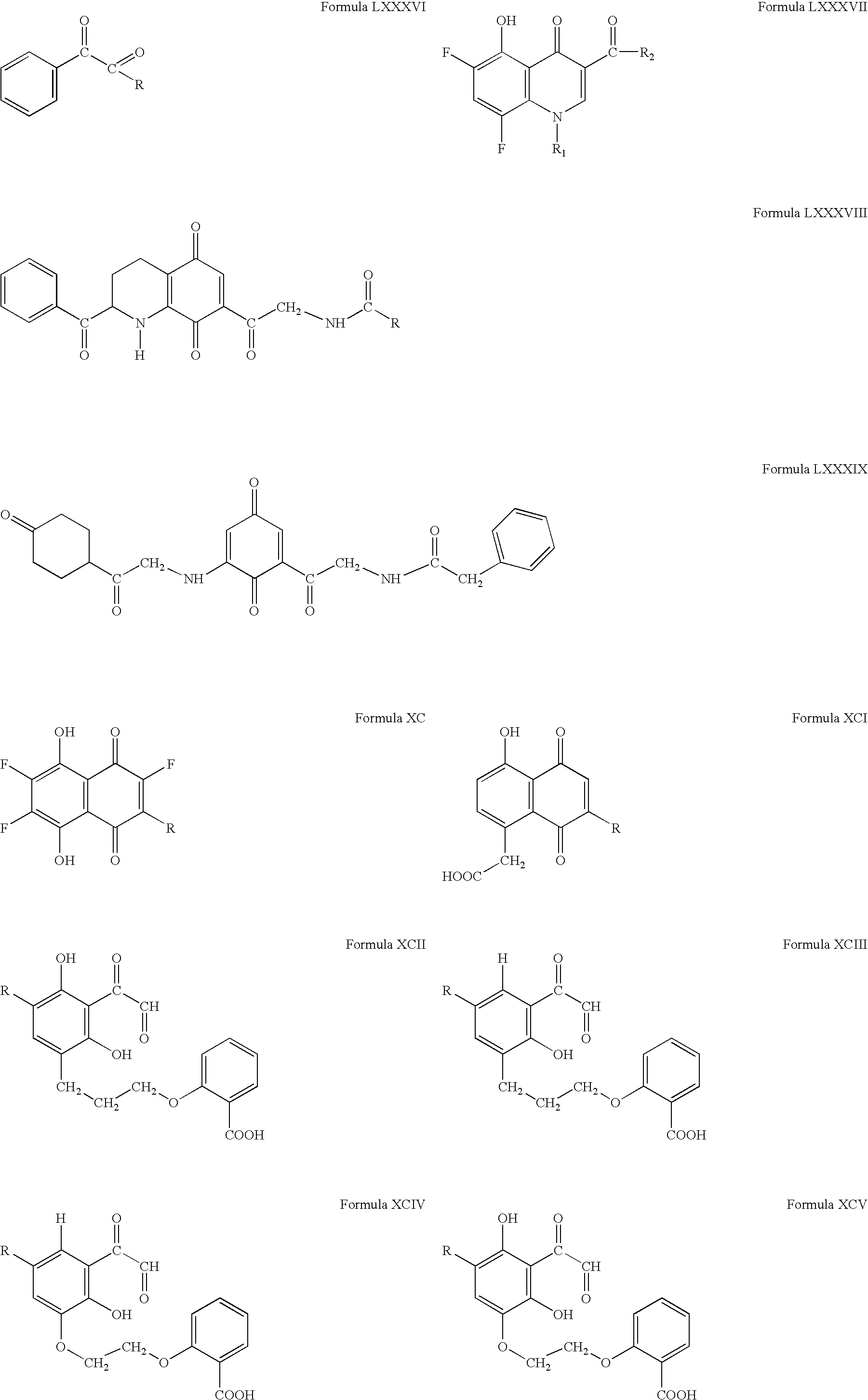

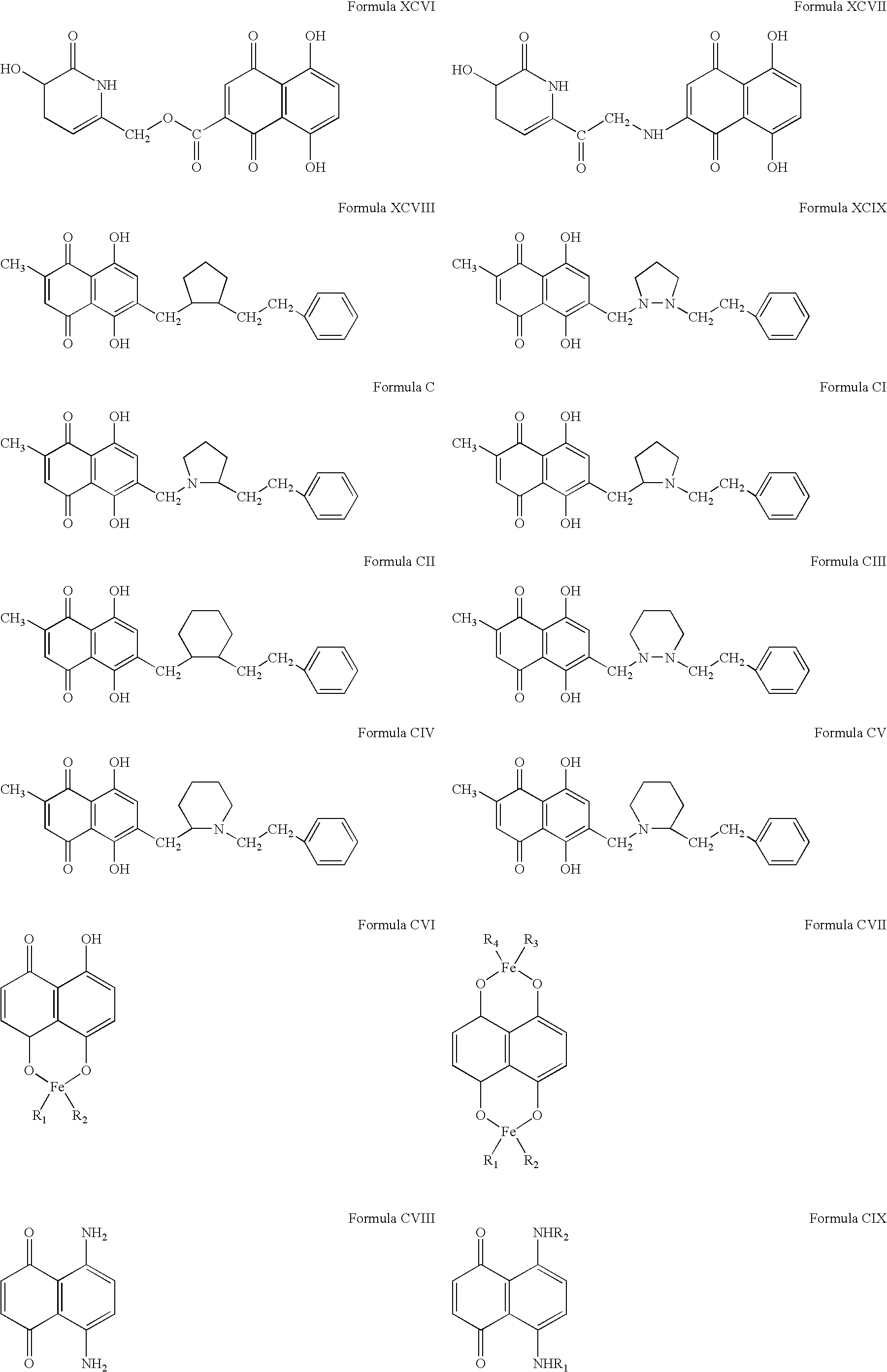

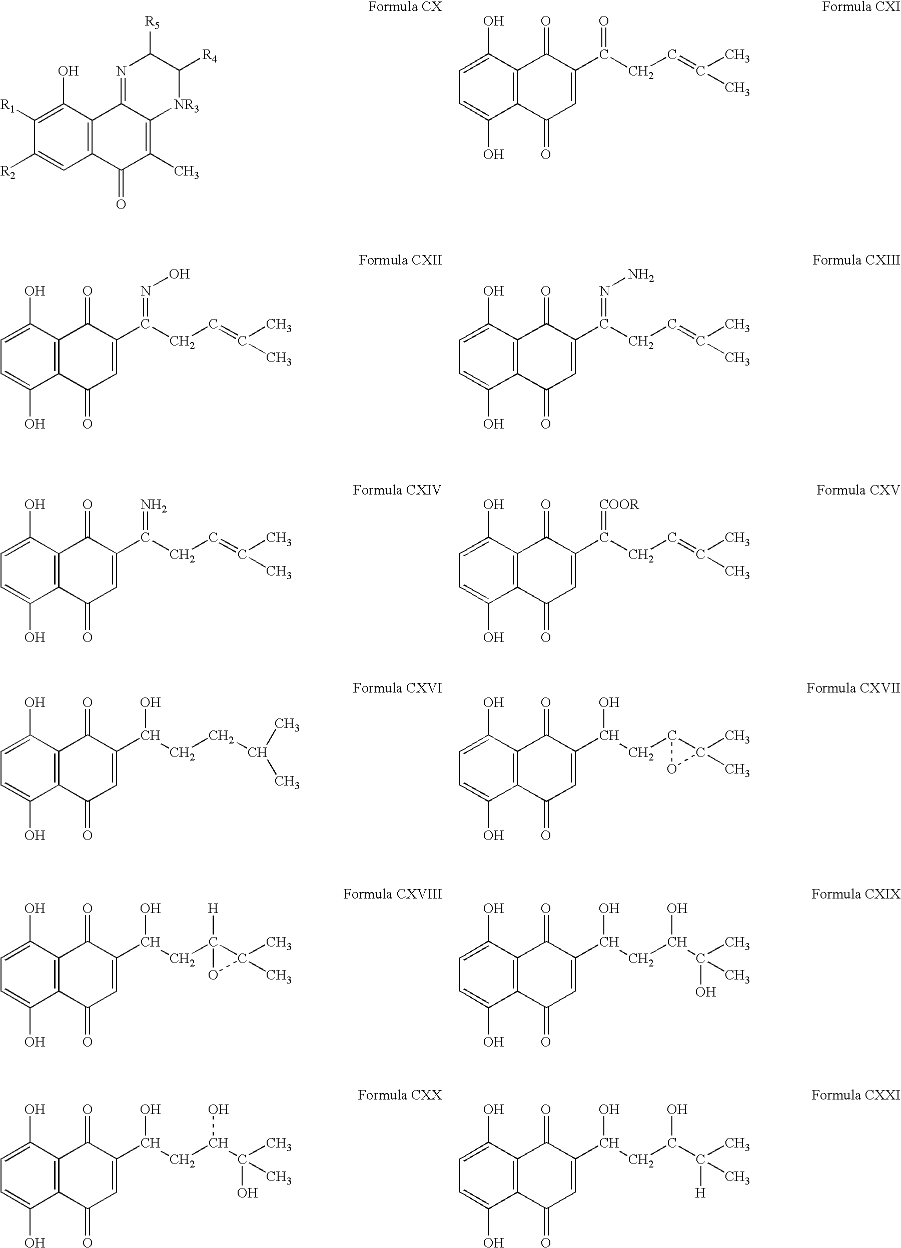

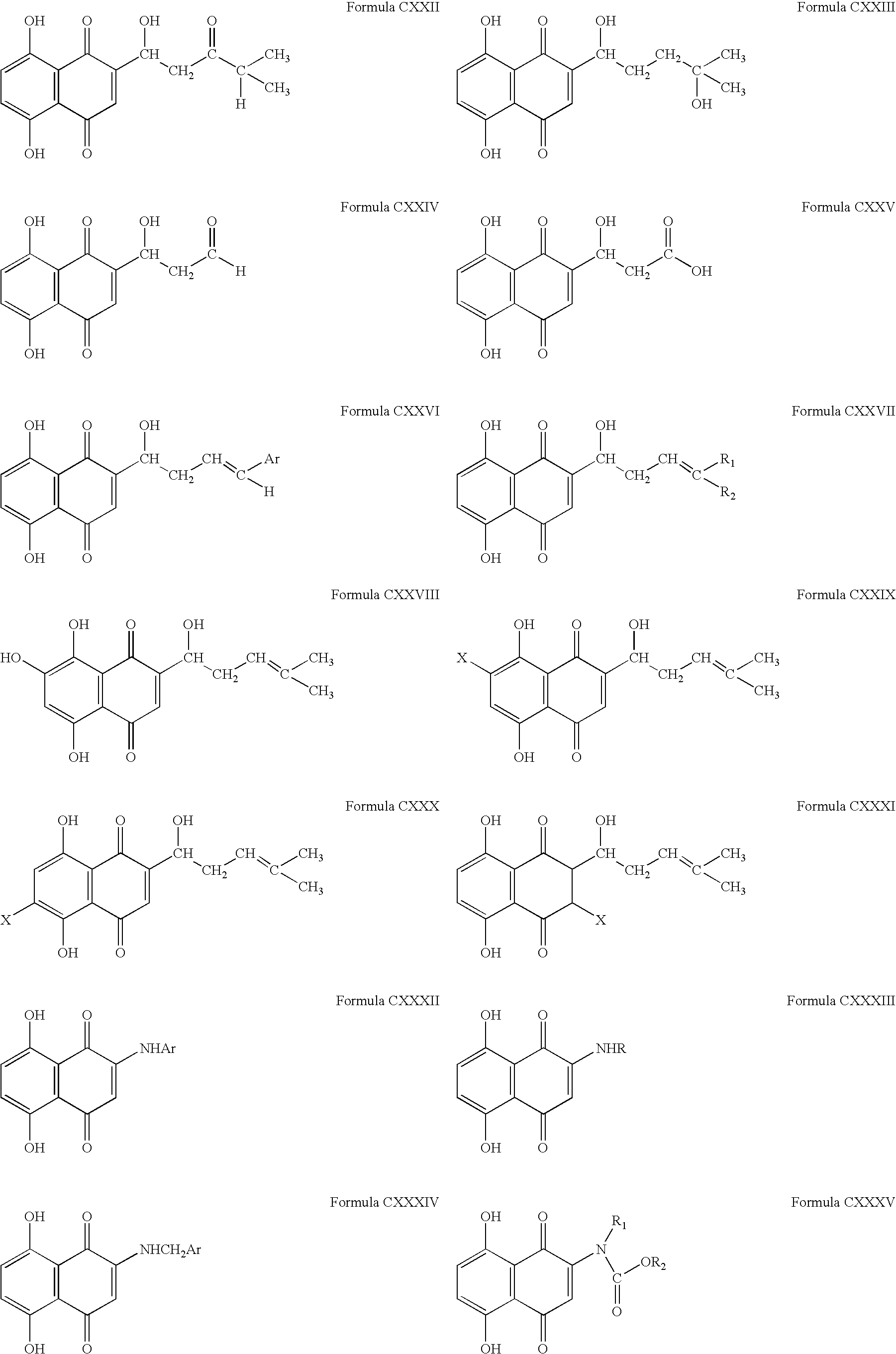

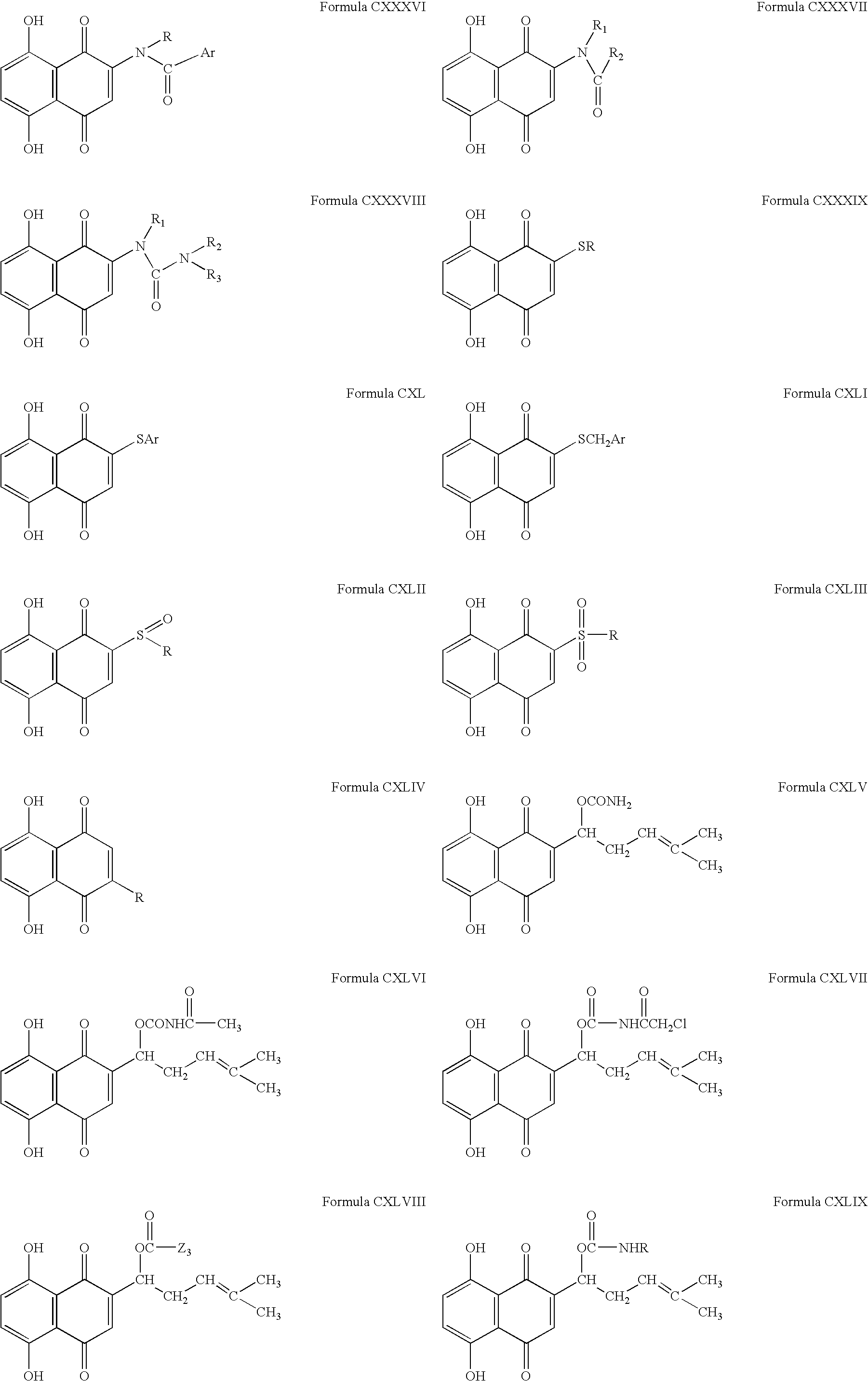

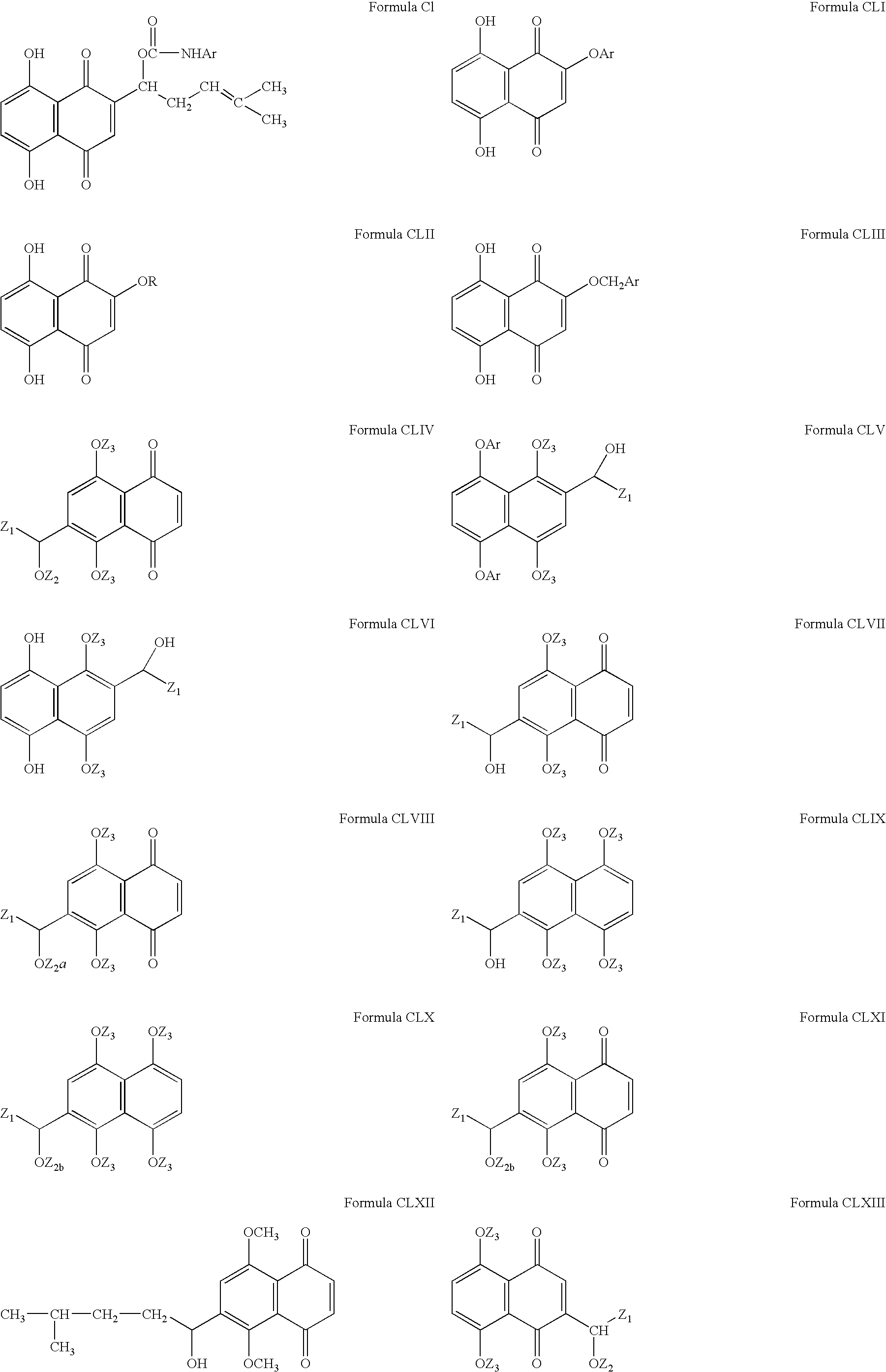

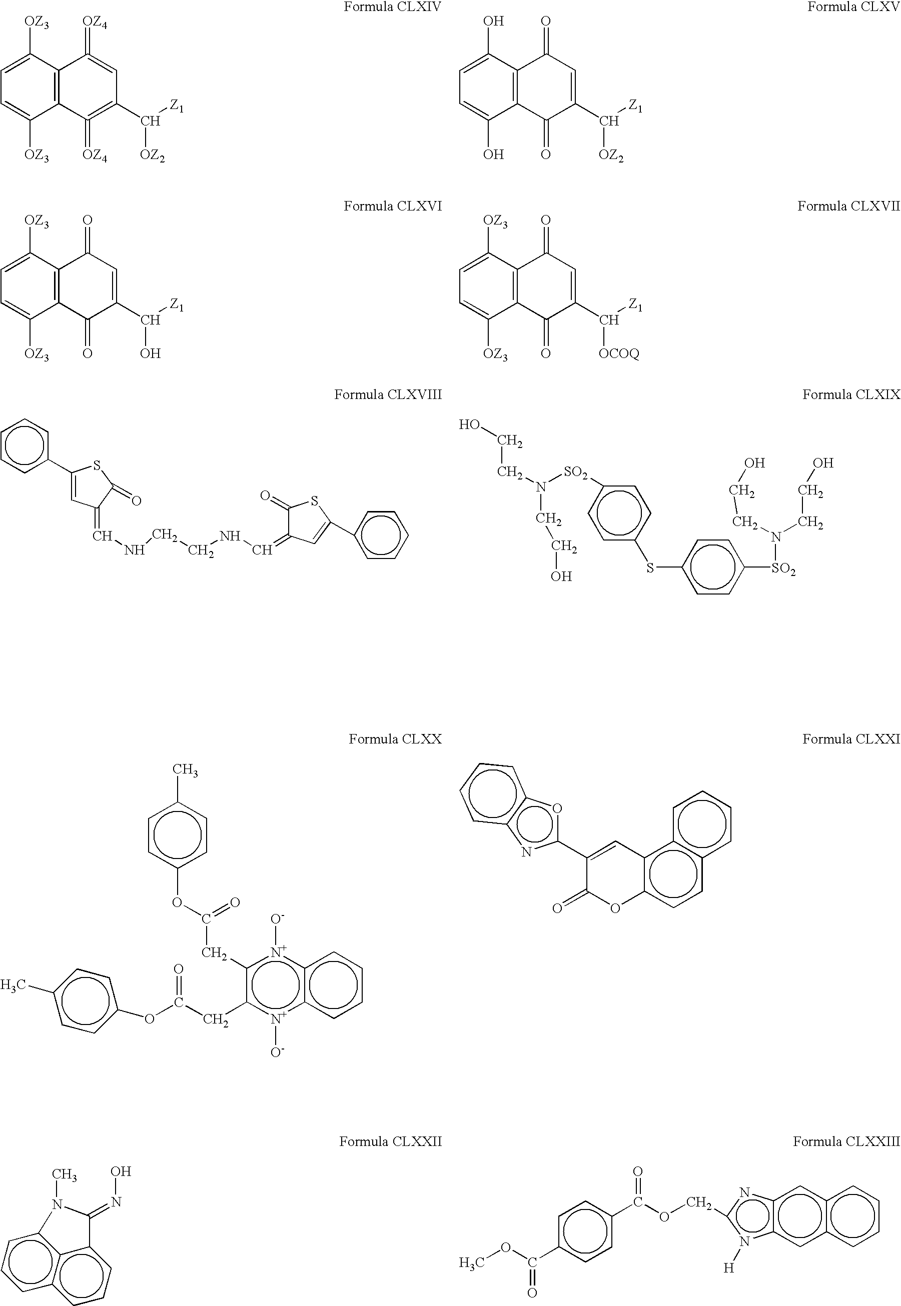

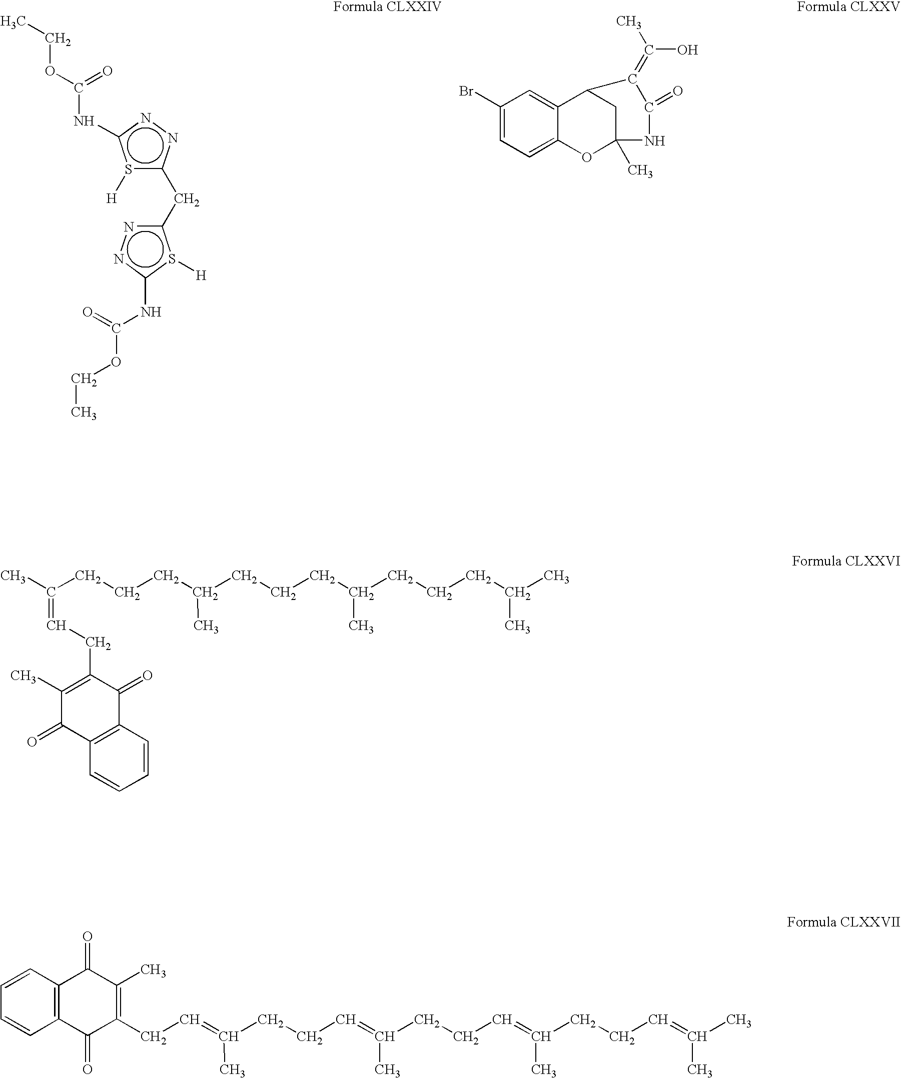

wherein A is one of the following

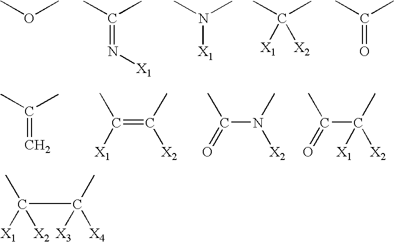

wherein T₁, T₂, T₃, T₄ are independently hydrogen, hydroxyl, halogen, methoxy, OCH₂COOH, OCH₂CONH₂, SO₂NH₂, NHSO₂NH₂, NH—Q₁, CH₂—Q1, O—Q₁, S—Q₁, C₁-C₆ alkyl with or without substitution, C₁-C₆ alkyl ether C₁-C₆ alkyl, phenyl optionally substituted with Q₁, C₃-C₁₀ cycloalkyl or bicycloalkyl optionally substituted with Q₁, C₁-C₃ alkyloxy, —NH—CO—NH₂, —NH—(3,5-dinitro-phenyl), —NH—(2,4-dinitro-phenyl) or BCl₃; with Q1 as defined above;

wherein R, R₁, R₂, R₃, and R₄, being the same or different, can be any organic moiety, including substituted or unsubstituted alkyl, peptide or peptide mimetic, that would fit the active site of a target cysteine protease such as caspase, e.g., caspase-3, caspase-7, caspase-8, and caspase-9;

wherein X is a halogen;

wherein Ar is a substituted or unsubstituted aryl;

wherein Z₁ is a saturated or unsaturated alkyl with or without substitution or alkenyl with or without substitution; Z₂ is hydrogen, saturated or unsaturated alkyl with or without substitution or acyl with or without substitution or a group —C(O)Q wherein Q is alkyl, alkenyl, aryl, aralkyl or aralkenyl with or without substitution; Z₂ₐ is acyl with or without substitution; Z_{2b} is a saturated or unsaturated alkyl with or without substitution; Z₃ is hydrogen or a saturated or unsaturated alkyl with or without substitution; and Z₄ is saturated or unsaturated alkyl with or without substitution; and

wherein any —OH group at the side chain C(2′) position can be alpha and beta stereochemistry.

According to the present invention, there are provided pharmaceutical preparations comprising a pharmaceutically acceptable carrier, at least one active cysteine protease inhibitor, wherein the active cysteine protease inhibitor has one of the backbone structures given in Formulae LIX-CLXXVII, and at least one of the following: DTT or a derivative, HSCH₂CH₂OHCH₂OHCH₂SH, GSH (glutathione), HOOCCH(NH₂)CH₂CH₂CONHCH(CH₂SH)CONHCH₂COOH, mycothiol (MT), any other sulfur-reducing agent, any adduct of naphthoquinone derivative and DTT or GSH or MT or any adduct with a different oxidation state (e.g., NO*⁻ or NOH*⁻), or

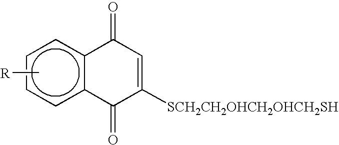

The backbones represented in Formulae I-CLXXVII are usefuil as cysteine protease inhibitors as predicted by caspase-3 inhibition studies and/or modeling results. Most of these compounds are Michael addition substrates, which are attacked by a deprotonated cysteine. The fact that most are cyclic compounds provides drug activity by holding the compound in the conformation that fits in the enzyme active site and by stabilizing the complex with the deprotonated cysteine by a conjugated pi system.

The active ingredients for the pharmaceutical preparations of the present invention can be synthesized according to methods well known in the art. In addition, they can be obtained commercially from Nanoscale Combinatorial Synthesis, Inc., (NANOSYN®; Mountain View, Calif.). The active ingredients for the pharmaceutical preparations of the present invention can be applied as drugs or pro-drugs or as any combination or derivative.

The pharmaceutical preparations of the invention are for the treatment of viral infections and of diseases wherein excessive apoptosis is implicated and/or wherein apoptosis should be reduced. In one aspect, the pharmaceutical preparations are suitable for treatment of 3C-protease modulated infectious diseases, neurodegenerative diseases and certain cardiovascular diseases, e.g., common colds, allergic rhinitis, poliomyelitis, hepatitis-A, encephalitis, meningitis, hand-foot-and-mouth disease, encephalomyocarditis, summer flu (enteroviral upper respiratory infection), asthma, various allergies, myocarditis, acute hemorrhagic conjunctivitis, disseminated neonatal infection and Borhnolm's disease. All the above are diseases which manifestation is dependent on the activity of a cysteine protease of the CB clan. The pharmaceutical preparations of the present invention are also suitable for the treatment of diseases manifested by the activity of the cysteine proteases of the CD clan, i.e. apoptosis-involved diseases, which are caused by excessive apoptosis, e.g., neurodegenerative diseases such as Alzheimer's, Parkinson's and Huntington's diseases; spinal muscular atrophy; multiple sclerosis; immune-based diseases such as immunodeficiency, hypersensitivity, and autoimmune disorders; ischemic cardiovascular and neurological diseases or injury such as stroke, myocardial infarction, spinal cord injury or transplant organ damage; inflammatory diseases such as arthritis, cholangitis, colitis, encephalitis, endocerolitis, hepatitis, pancreatitis and reperfusion injury; diabetes; and alopecia.

One preferred application of caspase-3 inhibitors is to minimize the amount of brain damage due to apoptosis, which occurs in the hours following a stroke. Preferred treatment methods for stroke victims become a function of both the pharmacokinetics of the drug and how quickly the patient was gotten to an emergency room. For this application, a preparation comprising a caspase-3 inhibitor that crosses the blood-brain barrier readily is injected into the patient's blood stream. For preparations comprising a caspase-3 inhibitor that does not cross the blood-brain barrier quickly, the preparation is preferably injected into the spinal fluid.

In another aspect, the present invention is a method for the treatment of infectious diseases or physiopathological diseases or disorders associated with the enzymatic activity cysteine proteases, in particular caspases or 3C proteases. The method of treatment comprises administrating to a subject in need of such treatment an effective, pharmaceutically acceptable amount of a compound having the backbone of Formula I-CLXXVII, which has cysteine protease inhibitor activity, optionally together with a pharmaceutically acceptable carrier.

Pharmaceutically acceptable carriers are well known in the art and are disclosed, for instance, in Sprowl's American Pharmacy, Dittert, L. (ed.), J.B. Lippincott Co., Philadelphia, 1974, and Remington's Pharmaceutical Sciences, Gennaro, A. (ed.), Mack Publishing Co., Easton, Pa., 1985.

Pharmaceutical preparations of the compounds of the present invention, or of pharmaceutically acceptable salts thereof, may be formulated as solutions or lyophilized powders for parenteral administration. Powders may be reconstituted by addition of a suitable diluent or other pharmaceutically acceptable carrier prior to use. The liquid formulation is generally a buffered, isotonic, aqueous solution, but a lipophilic carrier, such as propylene glycol optionally with an alcohol, can be more appropriate for compounds of this invention. Examples of suitable diluents are normal isotonic saline solution, standard 5% dextrose in water of buffered sodium or ammonium acetate solution. Such a formulation is especially suitable for parenteral administration, but can also be used for oral administration or contained in a metered dose inhaler of nebulizer for insufflation or spray or drops to the nasal mucosa. It may be desirable to add excipients such as ethanol, polyvinylpyrrolidone, gelatin, hydroxy cellulose, acacia, polyethylene glycol, mannitol, sodium chloride or sodium citrate.

Alternately, the compounds of the invention may be encapsulated, tableted or prepared in an emulsion or syrup for oral administration. Pharmaceutically acceptable solid or liquid carriers may be added to enhance or stabilize the preparations, or to facilitate preparation. Liquid carriers include syrup, soy bean oil, peanut oil, olive oil, glycerin, saline, ethanol, and water. Solubilizing agents, such as dimethylsulfoxide, ethanol or formamide, may also be added. Carriers, such as oils, optionally with solubilizing excipients, are especially suitable. Oils include any natural or synthetic non-ionic water-immiscible liquid, or low melting solid capable of dissolving lipophilic compounds. Natural oils, such as triglycerides are representative.

Solid carriers include starch, lactose, calcium sulfate dihydrate, terra alba, magnesium stearate or stearic acid, talc, pectin, acacia, agar or gelatin. Solubilizing agents, such as dimethylsulfoxide or formamide, may also be added. The carrier may also include a sustained release material such as glyceryl monostearate or glyceryl distearate, alone or with a wax. The pharmaceutical preparations are made following the conventional techniques of pharmacy involving milling, mixing, granulating, and compressing for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation can be administered directly p.o. or filled into a soft gelatin capsule.

For rectal administration, a pulverized powder of the compounds of this invention may be combined with excipients such as cocoa butter, glycerin, gelatin or polyethylene glycols and molded into a suppository. The pulverized posers may also be compounded with an oily preparation, gel, cream or emulsion, buffered or unbuffered, and administered through a transdermal patch.

As will no doubt be appreciated by the person skilled in the art, the above Formulae I-CLXXVII represent a large number of possible compounds, and some of the compounds are more effective inhibitors of cysteine proteases of the above types than others. In order to determine compounds having are suitable as cysteine protease inhibitors, prospective compounds can be screened for inhibitory activities according to one of the following exemplary assays.

In Vitro High Throughput Caspase-3 Assay

Purified Human recombinant caspase-3, fluorescence labeled substrate (Acetyl-Asp-Glu-Val-Asp-7-amino-4-trifluoromethyl coumarin), and known inhibitor (Z-Asp-Glu-Val-Asp-fluoromethyl ketone) can be purchased from Sigma Chemical Co. (St. Louis, Mo.). The enzyme reactions are carried out at room temperature in 10 mM PIPES pH=7.4, 2 mM EDTA, 0.1% CHAPS, 5 mM DTT reaction buffer. Each well on the 96 well plate contains the above reaction buffer plus 55 μM fluorescence labeled substrate, 0.125 μg caspase-3, and 20-250 μM test compound. Positive and negative controls are present on each assay plate. A standard curve is generated using 7-amino-4-trifluoromethyl coumarin in the concentration range from 0.15 μM to 5.0 μM. The reactions are preferably read every 0.5 hours using a fluorescence microtiter plate reader set for 390 nm excitation and 538 nm emission for the first 4 hours after which the plates are left at room temperature overnight. One reading is taken the next morning as a final measurement. Activity is reported as percentage of the positive control.

In vitro Assays for Screening 3C-Protease Inhibitors

Birch et al have developed a continuous fluorescence assay to determine kinetic parameters and to screen potential HRV14 3C protease inhibitors. The assay consists of a consensus peptide for rhinoviruses connected to a fluorescence donor group (anthranilic acid; Anc) at the N terminal and to an acceptor group (p-NO₂-Phe; Pnp) at the P4 position, both groups flanking the scissile bond (Gln/Gly). The substrate peptide consists of the following sequence: Anc-Thr-Leu-Phe-Gln-Gly-Pro-Val-Pnp-Lys. There is a linear time dependent increase in fluorescence intensity as the substrate is cleaved, which allows continuous monitoring of the reaction. Multiwell plates containing one inhibitor per well allows for rapid screening by measuring the fluorescence intensity in each well. (Birch et al. 1995. Protein Expression and Purification 6:609-618).

Heinz et al have developed an assay method for measuring 3C protease activity and inhibition using the substrate biotin-Arg-Ala-Glu-Leu-Gln-Gly-Pro-Tyr-Asp-Glu-Lys-fluorescein-isothiocyanate. Cleavage mixtures containing inhibitors are allowed to bind to avidin beads and are subsequently washed. The resultant fluorescence of the bead is proportional to the degree of inhibition. (Heinz et al. 1996. Antimicrobial Agents and Chemotherapy 40:267-270).

McCall et al developed an assay that measures in addition to the inhibitory effects of the candidate inhibitors, their capability to enter into cells so that a high capacity screen for compounds inhibiting the 3C protease of HRV-1B is developed. The assay uses a recombinant strain of E-coli expressing both the protease and a tetracycline resistance gene modified to contain the minimal 3C protease cleavage sequence. Cultures growing in microtiter plates containing tetracycline are treated with potential inhibitors. Culture with no inhibition of the 3C protease, show reduced growth due to cleavage of the essential gene product. Normal growth is seen only in cultures that contains an effective 3C protease inhibitor. (McCall et al. 1994. Bio/Technology 12:1012-1016).

An assay was developed in our lab based on a protein consisting of the 3C protease fused to DHFR. The cleavage of the fusion protein by external 3C protease (type 1A) is monitored by gel-electrophoresis. The degree of cleavage is proportional to the ratio of low molecular weight proteins (3 C and DHFR) to intact fusion protein, as observed on the gel.

The present invention will now be described in reference to some non-limiting examples. It is to be understood that the examples contain exemplary embodiments of the invention and is intended to be illustrative of the invention, but is not to be construed to limit the scope of the invention in any way.

### EXAMPLE 1

### Cell Culture 3C-Protease Activity Assay

In this assay, 96 well micro titer plates were seeded with 10⁴ HeLa-H1 (ATCC) cells per well and incubated in DMEM+10% FBS (Gibco) for 24 hours at 37° C., saturated humidity and 5% CO₂. Human Rhinovirus serotype 1A (ATCC) were titered to produce a 30% cell kill and added to some wells of a 96 well plate, other wells were mock infected with media only, followed by incubation at 33° C., saturated humidity and 5% CO₂ for 1 hour. Compounds were dissolved in DMSO, diluted in DMEM and added in a 9 step 2-fold dilution series (250, 125, 62.5, 31.25, 15.63, 7.81, 3.91, 1.95, 0.98 μM) 1 hour after virus treatment. Plates were incubated for 48 hours at 33° C., saturated humidity and 5% CO₂ in a final volume of 100 μL FBS free DMEM. Cell survival was measured by the addition of 10 μL of alamarBlue™ (BioSource), incubation at 33° C. for 45 minutes and reading in a fluorescence plate reader, excitation: 544 nm, emission: 590 nm. The inhibitory concentration 50% (IC₅₀) was calculated as the concentration of compound that increased the percentage fluorescence in the compound-treated virus-infected cells to 50% of that produced by compound-free, uninfected cells. The toxicity concentration 50% (TC₅₀) was calculated as the concentration of compound that decreased the percentage fluorescence in the compound-treated, uninfected cells to 50% of the compound-free, uninfected cells. The therapeutic index (Ti) was calculated by dividing the IC₅₀ by the TC₅₀.

TABLE I
3C Protease Cell Culture Assay Results

| Tracking Number | Structure | IC50 μM | TC50 μM |
|---|---|---|---|
| cpi0132 | backbone: Formula II | 932.4, | —, |
| | Q₁ = OH | — | — |
| | R₁ = methyl | | |
| | R₂ and Q₂-Q₄ = H | | |
| cpi0136.0009 | backbone: Formula IX | 0.2 | >30 |
| | Q₁ and Q₂ = OH | | |
| | Q₄-Q₈ = H | | |
| | Q₃ = —CH₂N(CH₂COOH)₂ | | |
| cpi0136.0012 | backbone: Formula IX | 30 | >30 |
| | Q₁ = OH | | |
| | Q₂-Q₃ and Q₅-Q₈ = H | | |
| | Q₄ = —NHCH₂CH₂COOH | | |
| cpi0136.NS131731 | backbone: Formula IX | 19.4, | —, |
| | Q₁ = NHCH₂CH₂COOCH₃ | — | — |
| | Q₄ = OH | | |
| | Q₂-Q₃ and Q₅-Q₈ = H | | |
| cpi0136.NS53780 | backbone: Formula XII(ii) | 8.3 | — |
| | R₁-R₂ and Q₅-Q₇ = H | | |
| | X₁ = NHCONH₂ | | |
| cpi0136.NS55588 | backbone: Formula XII(ii) | 4.6 | 22.2 |
| | R₁-R₂ and Q₅-Q₇ = H | | |
| | X₁ = NH-(3,5-dinitro- | | |
| | phenyl) | | |
| cpi0176 | backbone: Formula LVI | 22.8, | —, |
| | R₁ = COOH | — | — |
| | R₂ and Q₁-Q₂ = H | | |
| | X₁ = ethyl | | |
| | Q₃ = methyl | | |
| cpi0409 | backbone: Formula III | 103.1, | —, |
| | A = —O— | 96.8, | —, |
| | Q₁ = methoxy | —, | —, |
| | R₁ = R₂ and Q₂-Q₄ = H | — | — |

Values are given in micromolar concentrations, and the hyphens indicate no value was able to be calculated (i.e. the drug response did not cross the 50% point).

### EXAMPLE 2

### Caspase-3 Inhibitors

Representative compounds of the present invention were purchased as part of a combinatorial library from Nanoscale Combinatorial Synthesis, Inc. (NANOSYN®; Mountain View, Calif.). A number of other compounds were purchased individually from commercial sources (i.e., Compound TestNumbers cpi0116-cpi0135). A few compounds were custom synthesized (i.e., Compound Test Numbers cpi0139-cpi0141).

To determine the caspase inhibitory activity of these compounds, the in vitro high throughput caspase-3 assay presented herein was utilized. Purified human recombinant caspase-3, fluorescence labeled substrate (Acetyl-Asp-Glu-Val-Asp-7-amino-4-trifluoromethyl coumarin), and known inhibitor (Z-Asp-Glu-Val-Asp-fluoromethyl ketone) were purchased from Sigma. The enzyme reactions were carried out at room temperature in 10 mM PIPES pH=7.4, 2 mM EDTA, 0.1% CHAPS, 5 mM DTT reaction buffer. Each well on the 96 well plate contained the above reaction buffer plus 55 μM fluorescence labeled substrate, 0.125 μg caspase-3, and 20-250 μM test compound. Positive and negative controls were present on each assay plate. A standard curve was generated using 7-amino-4-trifluoromethyl coumarin in the concentration range from 0.15 μM to 5.0 μM. The reactions were read every 0.5 hours using a fluorescence microtiter plate reader set for 390 nm excitation and 538 nm emission for the first 4 hours after which the plates were left at room temperature overnight. One reading was taken the next morning as a final measurement. Activity was reported as percentage of the positive control. The assay results are presented in Table II, wherein the lower the percent activity, the better inhibitory capacity of the test compound. A negative percent activity indicates the test compound is a better caspase inhibitor than the control caspase inhibitor.

TABLE II
Caspase Inhibition of Representative Compounds

| Test Compound | | Percent |
|---|---|---|
| Number | Compound | Activity |
| cpi0136.0001 | backbone: Formula IX | −8.93778 |
| | Q₁ and Q₄ = OH | |
| | Q₂ = Cl | |
| | Q₃ = COOH | |
| | Q₅-Q₈ = H | |
| cpi0136.0002 | backbone: Formula II | −6.091043 |
| | Q₁ = OH | |
| | Q₂-Q₄ and R₁-R₂ = H | |
| cpi0136.0004 | backbone: Formula XII | −3.390609 |
| | Q₈ = OH | |
| | Q₅-Q₇ and R₁-R₂ = H | |
| | X₁ = —N—NH—CO—NH₂ | |
| cpi0136.0005 | backbone: Formula IX | −2.335555 |
| | Q₁ and Q₅-Q₈ = H | |
| | Q₂ = SO₃ | |
| | Q₃ and Q₄ = OH | |
| cpi0136.0006 | backbone: Formula IX | −2.294729 |
| | Q₁-Q₂ and Q₄ = OH | |
| | Q₃ and Q₅-Q₈ = H | |
| cpi0136.0007 | backbone: Formula IX | −0.8436907 |
| | Q₁ = OH | |
| | Q₂-Q₃ and Q₅-Q₈ = H | |
| | Q₄ = NH₂ | |
| cpi0136.0008 | backbone: Formula XII | −0.7150032 |
| | Q₈ = OH | |
| | Q₅-Q₇ and R₁-R₂ = H | |
| | X₁ = —N—(3,5-dinitro-phenyl) | |
| cpi0136.0009 | backbone: Formula IX | −0.4238712 |
| | Q₁-Q₂ = OH | |
| | Q₄-Q₈ = H | |
| | Q₃ = —CH₂N(CH₂COOH)₂ | |
| cpi0136.0010 | backbone: Formula II | 1.451982 |
| | Q₁ and Q₃ and R₂ = OH | |
| | Q₂ and Q₄ = H | |
| | R₁ = 3,4-dihydroxy-phenyl | |
| | (Formula LVII) | |
| cpi0136.0011 | backbone: Formula IX | 2.420164 |
| | Q₁ and Q₄ = OH | |
| | Q₂ and Q₅-Q₈ = H | |
| | Q₃ = —CH₂COOH | |
| cpi0136.0012 | backbone: Formula IX | 7.845539 |
| | Q₁ = OH | |
| | Q₂-Q₃ and Q₅-Q₈ = H | |
| | Q₄ = —NHCH₂CH₂COOH | |
| cpi0136.0014 | backbone: Formula III | 10.48272 |
| | A = O | |
| | Q₁ and Q₃ = OH | |
| | Q₂ and Q₄ and R₁ = H | |
| | R₂ = 2-(5-ethyl-furan ester) | |
| cpi0136.0015 | backbone: Formula III | 12.64829 |
| | A = O | |
| | Q₁ and Q₃ = OH | |
| | Q₂ and Q₄ and R₁ = H | |
| | R₂ = 6-(2,3-dihydro-benzo[1,4]dioxine) | |
| cpi0136.0016 | backbone: Formula III | 17.06794 |
| | A = O | |
| | Q₁-Q₃ = OH | |
| | Q₄ and R₂ = H | |
| | R₁ = 4-hydroxy-phenyl | |
| cpi0136.0017 | backbone: Formula XIII | 24.80114 |
| | Q₁-Q₄ and Q₆-Q₇ and Q₉-Q₁₀ = H | |
| | Q₅ = OH | |
| | Q₈ = CH₃ | |
| cpi0136.0020 | backbone: Formula XII | 36.97881 |
| | Q₈ = OH | |
| | Q₅-Q₇ and R₁-R₂ = H | |
| | X₁ = —N-(2,4-dinitro-phenyl) | |
| cpi0136.0021 | Formula LVIII | 44.11 |
| cpi0136.0024 | backbone: Formula IX | 51.3817 |
| | Q₁ = OH | |
| | Q₂ = NO₂ | |
| | Q₃ = CH₃ | |
| | Q₄ = —OCH₃ | |
| | Q₅-Q₈ = H | |
| cpi0136.0025 | backbone: Formula III | 54.1256 |
| | Q₁ = OH | |
| | A = O | |
| | R₁ = phenyl | |
| | R₂ and Q₂ and Q₄ = H | |
| | Q₃ = OCH₂COOCH₂-phenyl | |
| cpi0124 | backbone: Formula III | 51.49 |
| | A = O | |
| | Q₁ and Q₃ = OH | |
| | R₁ = phenyl | |
| | Q₂ and Q₄ and R₂ = H | |
| cpi0126 | backbone: Formula II | −0.30 |
| | Q₁-Q₄ and R₁-R₂ = H | |
| cpi0127 | backbone: Formula II | 1.32 |
| | R₂ = CH₃ | |
| | Q₁-Q₄ and R₁ = H | |
| cpi0128 | backbone: Formula II | 1.67 |
| | R₁-R₂ = Cl | |
| | Q₁-Q₄ = H | |
| cpi0130 | backbone: Formula IX | 45.72 |
| | Q₁ and Q₄ = OH | |
| | Q₂-Q₃ and Q₅-Q₈ = H | |
| cpi0131 | backbone: Formula II | −1.78 |
| | Q₁ and Q₄ = OH | |
| | Q₂-Q₃ and R₁-R₂ = H | |
| cpi0132 | backbone: Formula II | 10.26 |
| | Q₁ = OH | |
| | R₁ = CH₃ | |
| | Q₂-Q₄ and R₂ = H | |
| cpi0133 | backbone: Formula II | 48.29 |
| | R₂ = OH | |
| | Q₁-Q₄ = H | |
| | R₁ = —CH₂CH═C(CH₃)₂ | |
| cpi0137.0006 | Formula LIX | 54.47 |
| cpi0138.0001 | Formula LX | 22.68 |
| cpi0138.0002 | Formula LXI | 30.37 |
| cpi0138.0003 | Formula LXII | 39.00 |
| cpi0156.0034 | backbone: Formula LIII | −0.630328 |
| | Q₅ = Br | |
| | Q₇ = NHCH₂CH₂OCOCH₃ | |
| | Q₁-Q₄ and Q₆ = H | |
| cpi0156.0042 | backbone: Formula LIII | 11.08194 |
| | Q₇ = OC₆F₆ | |
| | Q₁-Q₆ = H | |
| cpi0156.0044 | backbone: Formula II | −1.376429 |
| | R₂ = Cl | |
| | R₁ = morpholine | |
| | Q₁-Q₄ = H | |
| cpi0156.0045 | backbone: Formula LIII | 3.338116 |
| | Q₅ = CONH-cyclohexyl | |
| | Q₁-Q₄ and Q₆-Q₇ = H | |
| cpi0156.0046 | backbone: Formula II | −1.771577 |
| | R₁ = —N(COCH₃)(CH₂-phenyl) | |
| | Q₁-Q₄ = H | |
| | R₂ = Cl | |
| cpi0156.0047 | backbone: Formula LIII | 28.62582 |
| | Q₅ = piperazinyl-phenyl | |
| | Q₁-Q₄ and Q₆-Q₇ = H | |
| cpi0156.0048 | backbone: Formula LIII | 18.82405 |
| | Q₇ = NH-phenyl | |
| | Q₅ = piperadinyl | |
| | Q₁-Q₄ and Q₆ = H | |
| cpi0156.0051 | backbone: Formula LIII | 6.782472 |
| | Q₇ = NH-phenyl | |
| | Q₅ = NHCH₃ | |
| | Q₁-Q₄ and Q₆ = H | |
| cpi0156.0052 | backbone: Formula LIII | 31.97115 |
| | Q₇ = NH-phenyl | |
| | Q₅ = N(CH₃)₂ | |
| | Q₁-Q₄ and Q₆ = H | |
| cpi0156.0054 | backbone: Formula LIII | −3.263774 |
| | Q₇ = OCH₃ | |
| | Q₁-Q₆ = H | |
| cpi0156.0055 | backbone: Formula LIII | −1.448315 |
| | Q₇ = O-(3-methyl-phenyl) | |
| | Q₁-Q₆ = H | |
| cpi0156.0056 | backbone: Formula LIII | 1.881723 |
| | Q₇ = Cl | |
| | Q₁-Q₆ = H | |
| cpi0156.D1 | Formula LXIII | 24.72 |
| cpi0156.E1 | Formula LXIV | 19.82 |
| cpi0156.A3 | Formula LXV | 1.50 |
| cpi0157.B2 | Formula LXVI | 50.76 |
| cpi0157.C3 | Formula LXVII | 36.41 |
| cpi0157.F9 | Formula LXVIII | 31.50 |
| cpi0157.F10 | Formula LXIX | 19.48 |
| cpi0159.C3 | Formula LXX | 33.47 |

Ki determinations were carried out using the above assay system. IC50 values were calculated from plots of Percent Activity vs. In[Inhibitor] at a fixed substrate concentration of 55 μM. Kᵢ(app) was calculated according to Equation 1:

$$Ki ( app ) = \frac{IC50}{1 + \frac{[ Substrate ]}{Km}} \qquad Eq . 1$$

The concentration of inhibitors ranged from 75 to 0.067 μM with each inhibitor concentration assayed in duplicate. The Km for the substrate was determined from a standard Lineweaver-Burke plot. The results of the Ki determinations calculated by this method are given in Table III below.

TABLE III
Ki Determinations

| Test | | | |
|---|---|---|---|
| Compound | | | |
| Number | Compound | IC50(μM) | Ki(app)(μM) |
| cpi0136.0002 | backbone: Formula II | 0.02 | 0.0026 |
| | Q₁ = OH | | |
| | Q₂-Q₄ and R₁-R₂ = H | | |
| cpi0190 | Formula LXXI | 0.03 | 0.0054 |
| cpi0136.0004 | backbone: Formula XII | 0.43 | 0.0682 |
| | Q₈ = OH | | |
| | Q₅-Q₇ and R₁-R₂ = H | | |
| | X₁ = —NNHCONH₂ | | |
| cpi0140 | backbone: Formula II | 3.17 | 0.5032 |
| | Q₁ and Q₄ = OH | | |
| | Q₂-Q₃ and R₁-R₂ = Br | | |
| cpi0136.0008 | backbone: Formula XII | 3.51 | 0.5567 |
| | Q₈ = OH | | |
| | Q₅-Q₇ and R₁-R₂ = H | | |
| | X₁ = —N-(3,5-dinitro-phenyl) | | |
| cpi0126 | backbone: Formula II | 4.04 | 0.6417 |
| | Q₁-Q₄ and R₁-R₂ = H | | |
| cpi0127 | backbone: Formula II | 4.37 | 0.6939 |
| | R₂ = CH₃ | | |
| | Q₁-Q₄ and R₁ = H | | |
| cpi0136.0006 | backbone: Formula IX | 4.80 | 0.7615 |
| | Q₁ and Q₂ and Q₄ = OH | | |
| | Q₃ and Q₅-Q₈ = H | | |
| cpi0136.0001 | backbone: Formula IX | 5.09 | 0.8072 |
| | Q₁ and Q₄ = OH | | |
| | Q₂ = Cl | | |
| | Q₃ = COOH | | |
| | Q₅-Q₈ = H | | |
| cpi0128 | backbone: Formula II | 6.62 | 1.0505 |
| | R₁-R₂ = Cl | | |
| | Q₁-Q₄ = H | | |
| cpi0136.0011 | backbone: Formula IX | 7.13 | 1.1320 |
| | Q₁ and Q₄ = OH | | |
| | Q₂ and Q₅-Q₈ = H | | |
| | Q₃ = —CH₂COOH | | |
| cpi0136.0012 | backbone: Formula IX | 7.82 | 1.2411 |
| | Q₁ = OH | | |
| | Q₂-Q₃ and Q₅-Q₈ = H | | |
| | Q₄ = —NHCH₂CH₂COOH | | |
| cpi0136.0005 | backbone: Formula IX | 8.90 | 1.4131 |
| | Q₁ and Q₅-Q₈ = H | | |
| | Q₂ = SO₃ | | |
| | Q₃ and Q₄ = OH | | |
| cpi0136.0007 | backbone: Formula IX | 11.17 | 1.7725 |
| | Q₁ = OH | | |
| | Q₂-Q₃ and Q₅-Q₈ = H | | |
| | Q₄ = NH₂ | | |
| cpi0162.B04 | Formula CLXVII | 16.78 | 2.66 |
| cpi0136.0024 | backbone: Formula IX | 19.23 | 3.0514 |
| | Q₁ = OH | | |
| | Q₂ = NO₂ | | |
| | Q₃ = CH₃ | | |
| | Q₄ = —OCH₃ | | |
| | Q₅-Q₈ = H | | |
| cpi0156.A3 | Formula LXV | 27.9 | 4.42 |
| cpi0141 | backbone: Formula II | 30.92 | 4.9081 |
| | Q₁ = OH | | |
| | R₁ = Br | | |
| | Q₂-Q₄ and R₂ = H | | |
| cpi0136.0009 | backbone: Formula IX | 38.78 | 6.1550 |
| | Q₁-Q₂ = OH | | |
| | Q₄-Q₈ = H | | |
| | Q₃ = —CH₂N(CH₂COOH)₂ | | |
| cpi0136.0010 | backbone: Formula II | 61.53 | 9.7648 |
| | Q₁ and Q₃ and R₂ = OH | | |
| | Q₂ and Q₄ = H | | |
| | R₁ = 3,4-dihydroxy-phenyl | | |
| | (Formula LVII) | | |
| cpi0136.0016 | backbone: Formula III | 73.87 | 11.7248 |
| | A = O | | |
| | Q₁-Q₃ = OH | | |
| | Q₄ and R₂ = H | | |
| | R₁ = 4-hydroxy-phenyl | | |
| cpi0159.G08 | Formula CLXVI | 77.23 | 12.26 |
| cpi0162.E09 | Formula CLXIV | 89.68 | 14.23 |
| cpi0157.F10 | Formula LXIX | 91.05 | 14.45 |
| cpi0157.F10 | Formula CLXX | 91.05 | 14.45 |
| cpi0162.B02 | Formula CLXV | 91.12 | 14.46 |
| cpi0137.0006 | Formula LIX | 92.0 | 14.60 |
| cpi0136.0017 | backbone: Formula XIII | 105.52 | 16.7466 |
| | Q₁-Q₄ and Q₆-Q₇ and | | |
| | Q₉-Q₁₀ = H | | |
| | Q₅ = OH | | |
| | Q₈ = CH₃ | | |
| cpi0162.A02 | Formula CLXIII | 138.44 | 21.97 |
| cpi0136.0021 | Formula LVIII | 170.0 | 27.00 |
| cpi0136.0014 | backbone: Formula III | 172.53 | 27.3829 |
| | A = O | | |
| | Q₁ and Q₃ = OH | | |
| | Q₂ and Q₄ and R₁ = H | | |
| | R₂ = 2-(5-ethyl-furan ester) | | |
| cpi0157.F9 | Formula LXVIII | 251.21 | 39.87 |
| cpi0157.F09 | Formula CLXVIII | 251.21 | 39.87 |
| cpi0136.0025 | backbone: Formula III | 255.01 | 40.4737 |
| | Q₁ = OH | | |
| | A = O | | |
| | R₁ = phenyl | | |
| | R₂ and Q₂ and Q₄ = H | | |
| | Q₃ = OCH₂COOCH₂-phenyl | | |
| cpi0157.B2 | Formula LXVI | 332.5 | 52.78 |
| cpi0157.B02 | Formula CLXIX | 332.52 | 52.78 |
| cpi0136.0020 | backbone: Formula XII | 341.63 | 54.2212 |
| | Q₈ = OH | | |
| | Q₅-Q₇ and R₁-R₂ = H | | |
| | X₁ = —N-(2,4-dinitro-phenyl) | | |
| cpi0139 | backbone: Formula II | 439.98 | 69.8299 |
| | Q₁ = OH | | |
| | Q₂ and R₁-R₂ = Br | | |
| | Q₃-Q₄ = H | | |
| cpi0157.C3 | Formula LXVII | 825.3 | 130.99 |
| cpi0136.0015 | backbone: Formula III | 1780.84 | 282.6426 |
| | A = O | | |
| | Q₁ and Q₃ = OH | | |
| | Q₂ and Q₄ and R₁ = H | | |
| | R₂ = 6-(2,3-dihydro- | | |
| | benzo[1,4]dioxine) | | |

### EXAMPLE 3

### Computational QSAR Prediction of Blood-Brain Permeability

To assess whether the compounds of the present invention might be useful in treating neurodegenerative diseases, a QSAR (Quantitative Structure-Activity Relationship) model was used to computationally predict the blood-brain barrier permeability for each compound. We developed the QSAR model using the MOE software package from the Chemical Computing Group. A set of 75 compounds with known blood-brain partition coefficients were obtained from the literature (Luco, J. M. 1999. J Chem Inf Comput Sci 39:396-404). A set of 15 descriptors available in this software package were chosen based on a principle component analysis. The QSAR equation was then obtained by linear regression. The resulting QSAR prediction equation reproduced the test set log BB data to an accuracy of RMSE=0.375975 and R²32 0.781358.

The results of the study are presented in Table IV as log BB values, i.e., the base ten log of the ratio of concentration of compound in the brain to concentration in the blood.

TABLE IV
Blood-brain Barrier Permeability for Test Compounds

| Test Compound | | |
|---|---|---|
| Number | Compound | log BB |
| cpi0136.0001 | backbone: Formula IX | −1.315 |
| | Q₁ and Q₄ = OH | |
| | Q₂ = Cl | |
| | Q₃ = COOH | |
| | Q₅-Q₈ = H | |
| cpi0136.0002 | backbone: Formula II | 0.078346 |
| | Q₁ = OH | |
| | Q₂-Q₄ and R₁-R₂ = H | |
| cpi0136.0004 | backbone: Formula XII | −0.35131 |
| | Q₈ = OH | |
| | Q₅-Q₇ and R₁-R₂ = H | |
| | X₁ = —N—NH—CO—NH₂ | |
| cpi0136.0005 | backbone: Formula IX | −0.10933 |
| | Q₁ and Q₅-Q₈ = H | |
| | Q₂ = SO₃ | |
| | Q₃ and Q₄ = OH | |
| cpi0136.0006 | backbone: Formula IX | −0.70949 |
| | Q₁-Q₂ and Q₄ = OH | |
| | Q₃ and Q₅-Q₈ = H | |
| cpi0136.0007 | backbone: Formula IX | −0.35563 |
| | Q₁ = OH | |
| | Q₂-Q₃ and Q₅-Q₈ = H | |
| | Q₄ = NH₂ | |
| cpi0136.0008 | backbone: Formula XII | −1.795 |
| | Q₈ = OH | |
| | Q₅-Q₇ and R₁-R₂ = H | |
| | X₁ = —N-(3,5-dinitro-phenyl) | |
| cpi0136.0009 | backbone: Formula IX | −2.8204 |
| | Q₁-Q₂ = OH | |
| | Q₄-Q₈ = H | |
| | Q₃ = —CH₂N(CH₂COOH)₂ | |
| cpi0136.0010 | backbone: Formula II | −2.7219 |
| | Q₁ and Q₃ and R₂ = OH | |
| | Q₂ and Q₄ = H | |
| | R₁ = 3,4-dihydroxy-phenyl | |
| | (Formula LVII) | |
| cpi0136.0011 | backbone: Formula IX | −1.5401 |
| | Q₁ and Q₄ = OH | |
| | Q₂ and Q₅-Q₈ = H | |
| | Q₃ = —CH₂COOH | |
| cpi0136.0012 | backbone: Formula IX | −0.58694 |
| | Q₁ = OH | |
| | Q₂-Q₃ and Q₅-Q₈ = H | |
| | Q₄ = —NHCH₂CH₂COOH | |
| cpi0136.0014 | backbone: Formula III | −0.96693 |
| | A = O | |
| | Q₁ and Q₃ = OH | |
| | Q₂ and Q₄ and R₁ = H | |
| | R₂ = 2-(5-ethyl-furan ester) | |
| cpi0136.0015 | backbone: Formula III | −0.90518 |
| | A = O | |
| | Q₁ and Q₃ = OH | |
| | Q₂ and Q₄ and R₁ = H | |
| | R₂ = 6-(2,3-dihydro-benzo[1,4]dioxine) | |
| cpi0136.0016 | backbone: Formula III | −2.1205 |
| | A = O | |
| | Q₁-Q₃ = OH | |
| | Q₄ and R₂ = H | |
| | R₁ = 4-hydroxy-phenyl | |
| cpi0136.0017 | backbone: Formula XIII | 0.51571 |
| | Q₁-Q₄ and Q₆-Q₇ and Q₉-Q₁₀ = H | |
| | Q₅ = OH | |
| | Q₈ = CH₃ | |
| cpi0136.0020 | backbone: Formula XII | −1.796 |
| | Q₈ = OH | |
| | Q₅-Q₇ and R₁-R₂ = H | |
| | X₁ = —N-(2,4-dinitro-phenyl) | |
| cpi0136.0021 | Formula LVIII | −0.815 |
| cpi0137.0006 | Formula LIX | −2.540 |
| cpi0138.0001 | Formula LX | −0.951 |
| cpi0138.0002 | Formula LXI | −2.608 |
| cpi0138.0003 | Formula LXII | −0.096 |
| cpi0156.D1 | Formula LXIII | 1.633 |
| cpi0156.E1 | Formula LXIV | 1.891 |
| cpi0156.A3 | Formula LXV | −2.217 |
| cpi0157.B2 | Formula LXVI | −1.907 |
| cpi0157.C3 | Formula LXVII | −2.071 |
| cpi0157.F9 | Formula LXVIII | −2.334 |
| cpi0157.F10 | Formula LXIX | −0.603 |
| cpi0159.C3 | Formula LXX | 0.136 |

### EXAMPLE 4

### Cross-Reactivity

To evaluate whether inhibition was specific to caspase-3 or applicable to other cysteine proteases or other proteases, inhibitor molecules were assayed for their capacity to inhibit additional non-caspase proteases. Three proteases, TPCK-trypsin, α-chymotrypsin and papain, were used to test for protease inhibition cross-reactivity. Protease inhibition assays were performed in 96-well flat-bottomed micro-titer plates with the QuantiCleave™ protease assay kit (Pierce, Rockford, Ill.) according to the manufacturer's instructions. Briefly, the assay conditions contained 100 μM compound, 2 mg/mL of succinylated-casein and 0.15 mg/mL of the protease. The assay incubated in a final volume of 150 μLs 0.05 M sodium borate pH 8.5 buffer (NaB-buffer) for 20 minutes at 25° C. After the protease reaction, 50 μLs of a trinitrobenzenesulfonic acid (TNBSA) solution (1:15, TNBSA:NaB-buffer) was added to each reaction and further incubated for 20 minutes at 25° C. Absorbance values at 450 nm were read in an Emax (Molecular Devices, Sunnyvale, Calif.) micro plate reader. Appropriate controls were performed, including known inhibitors (soybean trypsin inhibitor, aminoethyl-benzenesulfonic acid and leupeptin) for the proteases.

Relative activities in the presence of inhibitor were calculated by taking the ratio of absorbance for a reaction containing inhibitor to the absorbance for a reaction without inhibitor. Significant inhibition was determined by testing the null hypothesis of equivalence between the mean absorbance of reactions containing inhibitor and the mean absorbance of reactions without inhibitor (2 tailed t-test, 2 df).

The results are given in Table V in terms of cross-reactivities, relative activity and significant difference of 100 μM protease inhibitors on the proteases trypsin, chymotrypsin and papain.

TABLE V
Caspase Inhibitor Cross-reactivity

| Protease | | | TPCK- | | α-Chy- | | Papain | |
|---|---|---|---|---|---|---|---|---|
| inhib- | Caspase-3 | | Trypsin | | motrypsin | | Ac- | |
| itor | Activity^{a} | P^{b} | Activity | P | Activity | P | tivity | P |
| STI | | | 0.017 | 0.003 | | | | |
| AEBSF | | | | | 0.466 | 0.041 | | |
| Leu- | | | | | | | 0.095 | 0.002 |
| peptin | | | | | | | | |
| cpi0002 | 0.01499 | | 0.635 | 0.029 | 0.823 | — | 0.128 | 0.006 |
| cpi0118 | | | 0.998 | —^{c} | 0.988 | — | 0.935 | — |
| cpi0131 | −0.0178 | | 0.819 | — | 0.915 | — | 0.505 | 0.016 |
| cpi0132 | 0.103 | | 0.644 | 0.042 | 0.857 | — | 0.500 | 0.023 |
| cpi0076 | 0.756 | | 1.044 | — | 1.079 | — | 0.935 | — |
| cpi0077 | 0.811 | | 1.058 | — | 1.093 | — | 0.912 | — |
| cpi0124 | 0.515 | | 1.075 | — | 1.029 | — | 0.445 | 0.010 |
| cpi0133 | 0.483 | | 1.196 | — | 1.081 | — | 0.897 | — |

^{a}Activity is defined as: (Abs₄₅₀ₙₘ with inhibitor)/(Abs₄₅₀ₙₘ without inhibitor).
^{b}P is probability from a 2-tailed t-test for rejecting the H₀: (Abs₄₅₀ₙₘ with inhibitor) = (Abs₄₅₀ₙₘ without inhibitor).
^{c}—is P ≧ 0.05, values where P < 0.05 are given; STI: Soybean Trypsin Inhibitor; AEBSF: aminoethylbenzenesulfonic acid;

### EXAMPLE 5

### 3C Protease Biochemical Assay

Human Rhinovirus serotype 1A (ATCC) was used to clone the 3C Protease into the expression vector pET16-b and transformed for production into the E. coli strain BL21-DE3-pLys-S. 3C Protease expression was induced with 1 mM IPTG at 25° C. and purified from the soluble protein extract by chromatography on a SourceQ (Pharmacia) followed by gel filtration. HRV 3CP activity was measured by fluorescence resonance energy transfer using a dimodified decapeptide substrate MOC-Arg-Ala-Glu-Leu-Gln-Gly-Pro-Tyr-Asp-Lys-DNP-NH₂ (7-methoxy coumarin-4-acetic acid fluorochrome and dinitrophenol quencher) with a Kₘ value of 16.8 μM. Inhibition was measured as a change in initial velocity (V₀) as a function of inhibitor (I) concentration and substrate (S) concentration. Assays were performed in 100 μL volumes in a 96 well format at 30° C. containing 25 mM Tris HCl pH 8.0, 150 mM NaCl, 1 mM EDTA pH 8.0, 6 mM DTT, 2-6uM substrate, 2% DMSO, 416 nM 3CP and inhibitor as needed. Fluorescence was monitored by excitation at 328 nm and emission at 393 nm with 10 nm cutoffs. Data were analyzed with the nonlinear regression analysis program EnzFitter (BioSoft) with the equation:

Kᵢ=(I/((VₘₐₓxS)/V₀)/Kₛ)−I−S

Other groups used other methods to calculate Ki, and the results vary with each method see another method for example: Webber et al. 1996. “Design, synthesis and evaluation of nonpeptidic inhibitors of human Rhinovirus 3C protease,” J Med Chem 39:5072-5082. For reference purposes, we have synthesized compounds no. 14 in this paper, namely, an isatin derivative, which exhibits a very similar IC₅₀ to our compound no cpi0176 (nalidixic acid). The Ki calculations, though, are different.

Substrate concentrations used were lower than the Kₘ of the substrate (16.8 uM) so no corrections for an S/Kₘ term were used.

TABLE VI
3C Protease Biochemical Assay Results

| Tracking | | Ki |
|---|---|---|
| number | Structure | μM |
| CPI0118 | backbone: Formula IV(i) | 80 |
| | R₂ and Q₂-Q₃ = OH | |
| | R₁, R₁₆, and Q₄ = H | |
| CPI0123 | backbone: Formula II | 396 |
| | R₁ = OH | |
| | Q₁-Q₄ = H | |
| | R₂ = 2′-3-hydroxy-[1,4]naphthoquinone | |
| CPI0126 | backbone: Formula II | 515 |
| | R₁-R₂ and Q₁-Q₄ = H | |
| CPI0127 | backbone: Formula II | 242 |
| | R₁ = methyl | |
| | R₂ and Q₁-Q₄ = H | |
| cpi0131 | backbone: Formula II | 307 |
| | Q₁ and Q₄ = OH | |
| | R₁-R₂ and Q₂-Q₃ = H | |
| cpi0132 | backbone: Formula II | 128 |
| | Q₁ = OH | |
| | R₁ = methyl | |
| | R₂ and Q₂-Q₄ = H | |
| cpi0139 | backbone: Formula II | 154 |
| | R₁-R₂ and Q₂ = Br | |
| | Q₁ = OH | |
| cpi0141 | backbone: Formula II | 73 |
| | Q₁ = OH | |
| | R₁ = Br | |
| | R₂ and Q₂-Q₄ = H | |
| CPI0176 | backbone: Formula LVI | 195 |
| | R₁ = COOH | |
| | R₂ and Q₁-Q₂ = H | |
| | X₁ = ethyl | |
| | Q₃ = methyl | |

If the calculation for Ki were made using Equation 1 on Page 56, the values for Ki will generally be 10-fold lower than those shown in Table IV.

1. A cysteine protease inhibitor of the formula: 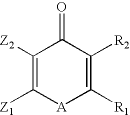
wherein
A is one of the following 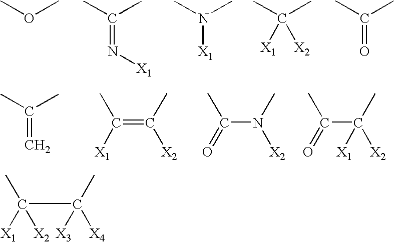
X₁, X₂, X₃, X₄ are independently hydrogen, hydroxyl, halogen, methoxy, OCH₂COOH, OCH₂CONH₂, SO₂NH₂, NHSO₂NH₂, NH—Q₁, CH₂—Q₁, O—Q₁, S—Q₁, C₁-C₆ alkyl, C₁-C₆ alkyl ether C₁-C₆ alkyl, phenyl optionally substituted with Q₁, C₃-C₁₀ cycloalkyl or bicycloalkyl optionally substituted with Q₁, C₁-C₃ alkyloxy, —NH—CO—NH₂, —NH-(3,5-dinitro-phenyl), —NH (2,4-dinitro-phenyl) or BCl₃;
R₁ and R₂ are independently hydrogen, hydroxyl, —COOH, 2-(5-ethyl-furan ester), 6-(2,3-dihydro-benzo[1,4]dioxine), halogen, SCH₂CH₂OH, CH₂CH₂OCH₃, morpholine, C₁-C₄ alkyl optionally substituted with R₁₀, C₂-C₄ alkenyl optionally substituted with R₁₀, or C₂-C₃ allylyl optionally substituted with R₁₀, CF₂—R₁₀, —O-phenyl optionally substituted with R₁₀, —S-phenyl optionally substituted with R₁₀, —CH₂-phenyl optionally substituted with R₁₀, —CH₂CH═C(CH₃)₂, NH—phenyl, dinethyl amine, methyl amine, 3-hydroxy-5-oxo-tetrahydro-furan-2-yl, —NH—CH₂-phenyl optionally substituted with R₁₀, benzene sulfinyl optionally substituted with R₁₀ wherein:
R₁₀ is halogen, hydroxy, methyl, ethyl, acetyl, carboxamide, nitro, sulfamido, phenyl or sulfamyl;
alternatively, R₁ and R₂ can form a C₃-C₁₀ cycloalkyl or bicycloalkyl, optionally containing 1 to 3 heteroatoms, optionally containing 1-3 unsaturations, and optionally substituted with hydrogen, hydroxy, halogen, amino, nitro, cyano, C₁-C₃ alkyl optionally substituted with 1-3 R₁₁, aryl ether optionally substituted with 1-5 R₁₁, CH₂OH, CH₂SH, CF₃, CONR₁₃R₁₄, SO₂NR₁₃R₁₄, SONR₁₃R₁₄, or NR₁₅ (C═O)R₁₄, wherein
R₁₁ is selected from the group consisting of halogen, cyano, nitro, amino, oxo, hydroxy, adamantyl, carbamyl, carbamyloxy, acetyl, C₁-C₄ alkyl optionally substituted with R₁₂, C₂-C₄ alkenyl optionally substituted with R₁₂, C₂-C₃ alkylyl optionally substituted with R₁₂, C₁-C₃ alkoxy optionally substituted with R₁₂, C₃-C₈ cycloalkyl optionally substituted with R₁₂, wherein:
R₁₂ is hydrogen, halogen, hydroxy, methyl, ethyl, acetyl, carboxamide, nitro, sulfamido, phenyl or sulfamyl;
R₁₃ is hydrogen or hydroxy;
R₁₄ is hydrogen, phenyl, benzyl, C₁-C₆ alkyl and C₃-C₆ cycloalkyl;
R₁₅ is hydrogenj hydroxyl, C₁-C₄ alkyl or benzyl;
Z₁ and Z₂ are hydrogen; or
alternatively Z₁ and Z₂ can form a C₁-C₅ cycloalkyl, optionally containing 1 to 3 heteroatoms, optionally containing 1-3 unsaturations, and optionally substituted with hydrogen, hydroxy, halogen, amino, nitro, cyano, C₁-C₃ alkyl optionally substituted with 1-3 R₁₁, C₁-C₃ alkoxy optionally substituted with 1-3 R₁₁, aryl ether optionally substituted with 1-5 R₁₁, CH₂OH, CH₂SH, CF₃, CONR₁₃R₁₄, SO₂NR₁₃R₁₄, SONR₁₃R₁₄,
NR₁₅(C═O)R₁₄, wherein R₁₁, R₁₂, R₁₃, R₁₄, and R₁₅ are as defined above, and wherein when A is O═C—N or C═C, A can be optionally substituted with R₁₆ and R₁₇, wherein
R₁₆ and R₁₇ are hydrogen, methyl, ethyl, isopropyl, halogen, phenyl, hydroxy, nitro, sulfamyl, or acetyl; or
alternatively Z₁ and Z₂ can form a heterocyclic ring system having a C₆-C₇ cycloalkyl fused to an aromatic ring, wherein the aromatic ring optionally containing 1 to 3 heteroatoms, optionally containing 1-3 unsaturations, and optionally substituted with hydrogen, hydroxy, halogen, amino, nitro, cyano, C₁-C₃ alkyl optionally substituted with 1-3 R₁₁, C₁-C₃ alkoxy optionally substituted with 1-3 R₁₁, aryl ether optionally substituted with 1-5 R₁₁, CH₂OH, CH₂SH, CF₃, CONR₁₃R₁₄, SO₂NR₁₃R₁₄, SONR₁₃R₁₄, or NR₁₅(C═O)R₁₄, wherein R₁₁, R₁₂, R₁₃, R₁₄, and R₁₅ are as defined above, and wherein when A is O═C—N or C═C, A can be optionally substituted with R₁₆, R₁₇, and R₁₈ wherein
R₁₆, R₁₇, and R₁₈ are hydrogen, methyl, ethyl, isopropyl, halogen, phenyl, hydroxy, nitro, sulfamyl, or acetyl; and
Q₁-Q₁₂ are hydrogen, hydroxyl, halogen, carboxylic acid, aldehyde, unsubstituted or substituted carboxylic acid, phenyl, benzyl, amide, amine, peptide, peptidomiimetic, t-butyl, isopropyl, methyl, ethyl, SO₃, NH₂, CH₂-COOH, nitro, NH—CH₂—CH₂—COOH, O-cyclopropyl-NHCOCH₂CH₂COOH, CH₂-cyclopropyl-NHCOCH₂CH₂COOH, NH-cyclopropyl-NHCOCH₂CH₂COOH, OCH₂CH₂NHCOCH₂CH₂COOH, CH₂CH₂CH₂NHCOCH2CH₂COOH, NHCH₂CH₂NHCOCH₂CH₂COOH, O-cyclopropyl-CH₂COCH₂CH₂COOH, CH₂-cyclopropyl-CH₂COCH₂CH₂COOH, NH-cyclopropyl-CH₂COCH₂CH₂COOH, OCH₂CH₂CH₂COCH₂CH₂COOH, CH₂CH₂CH₂CH₂COCH₂CH₂COOH, NHCH₂CH₂CH₂COCH₂CH2COOH, O-cyclopropyl-CH₂COCH₂CH₂Q₁, CH₂-cyclopropyl-CH₂COCH2CH₂Q₁, NH-cyclopropyl-CH₂COCH₂CH₂Q₁, OCH₂CH₂CH₂COCH₂CH₂Q₁, CH₂CH₂CH₂CH₂COCH₂CH₂Q₁, NHCH₂CH₂CH₂COCH₂CH₂Q₁, —NHCH₂CH₂COOCH₃, —CH₂N(CH₂COOH)₂, piperazinyl, or piperadinyl.

2. The cysteine protease of claim 1, wherein said compound has a backbone structure selected from the group consisting of:

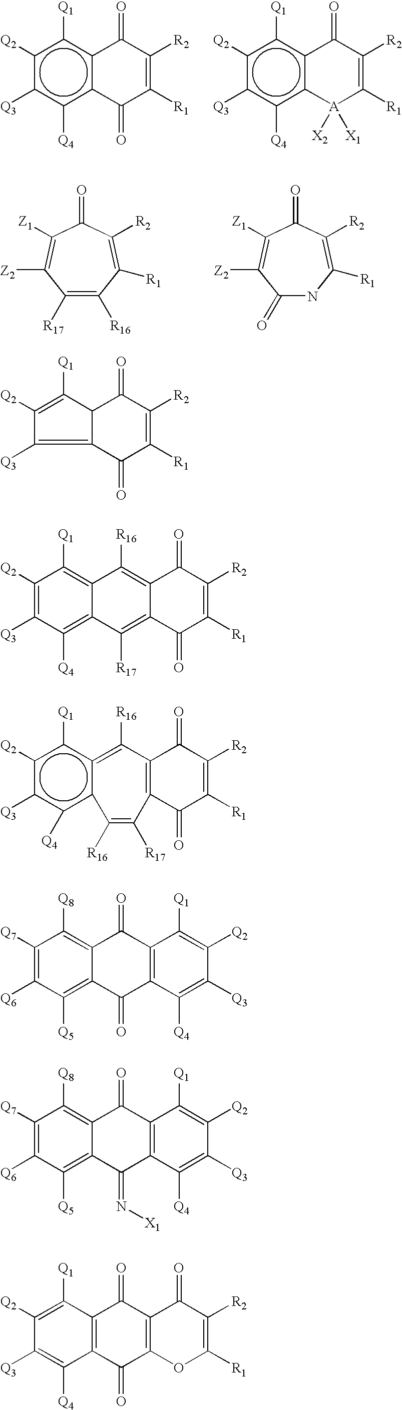

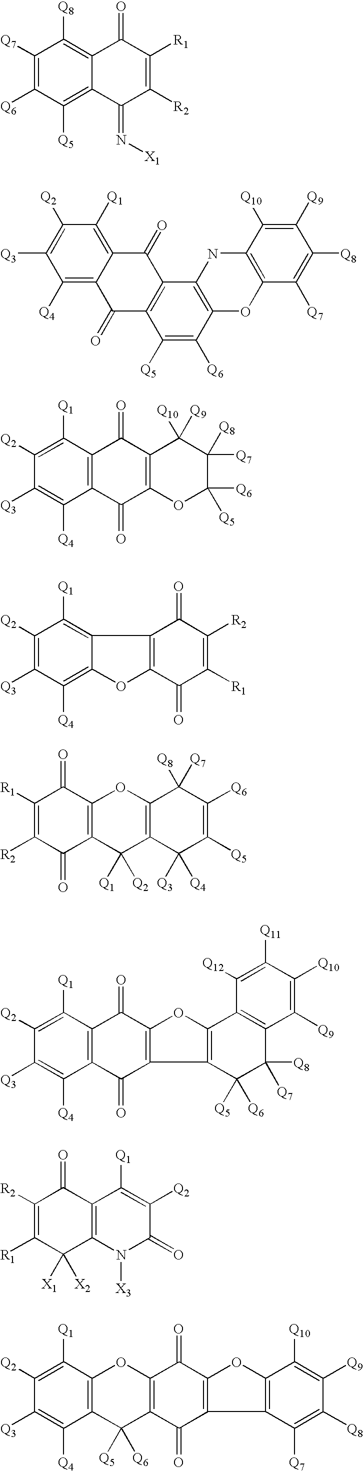

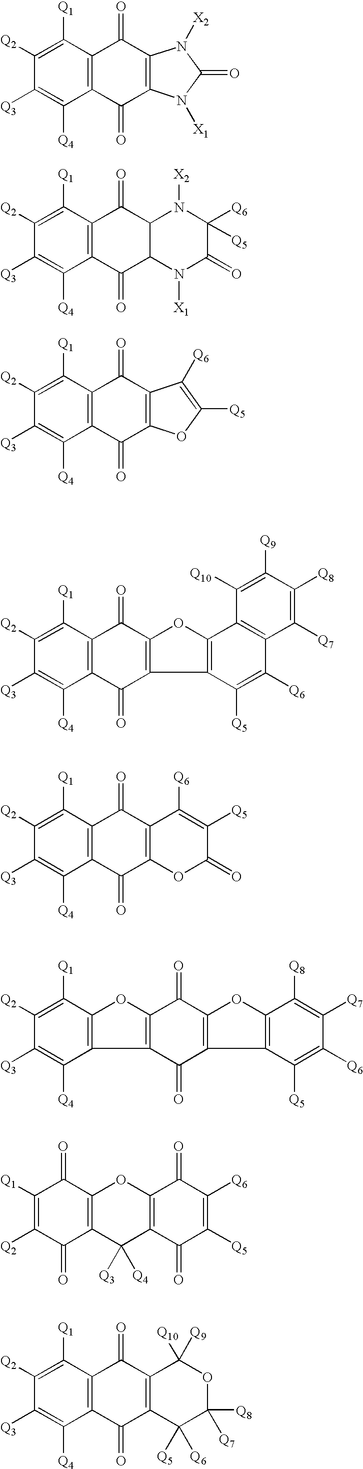

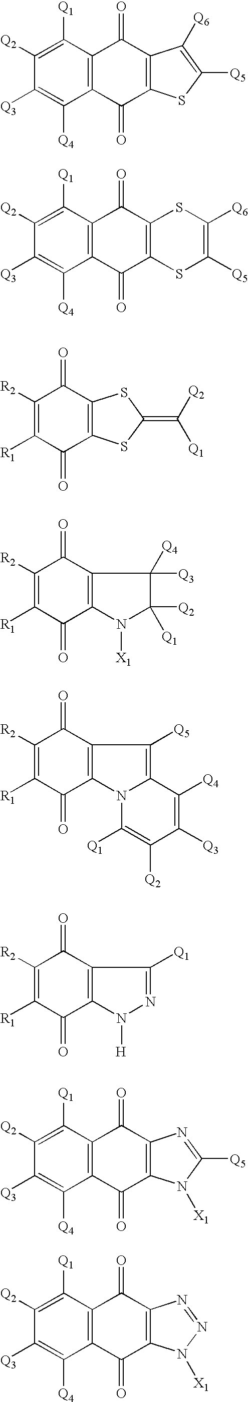

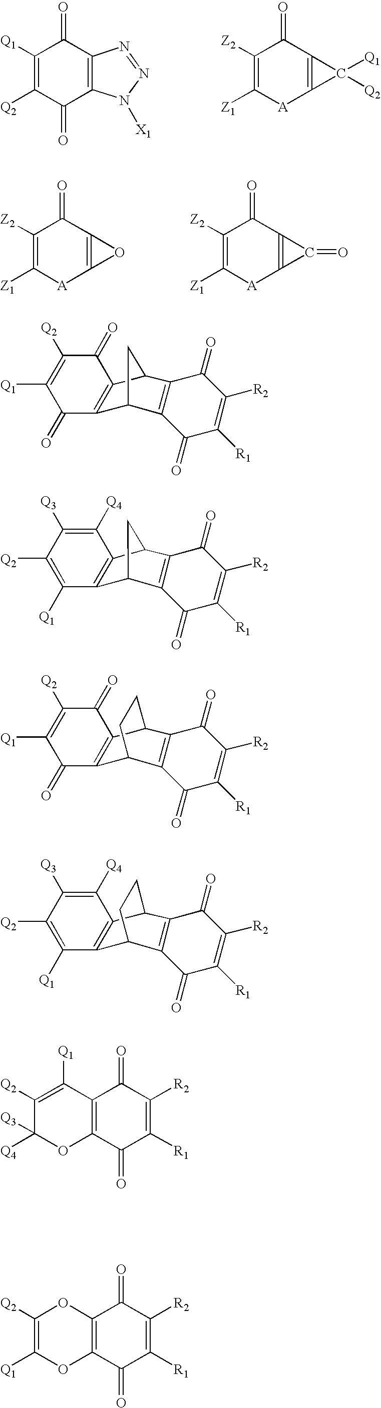

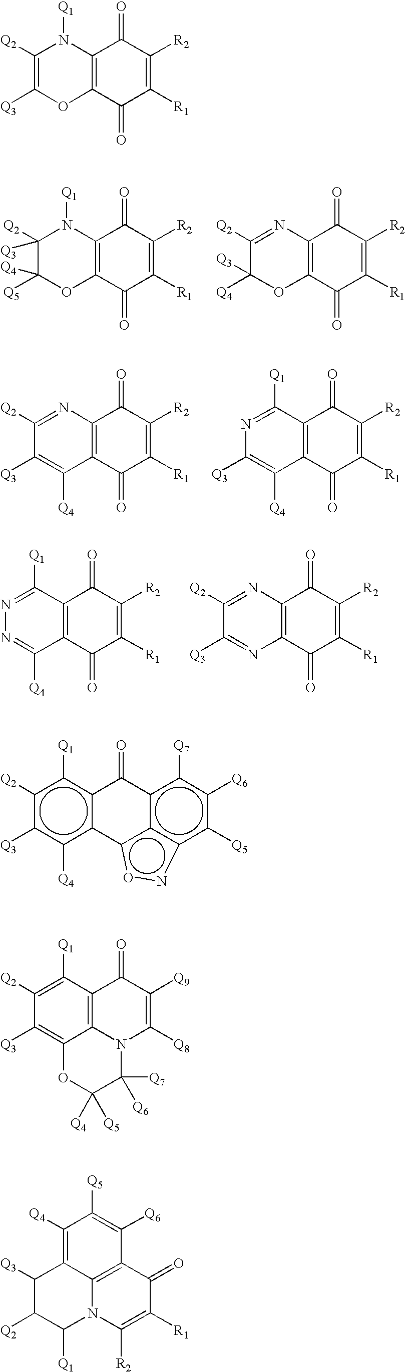

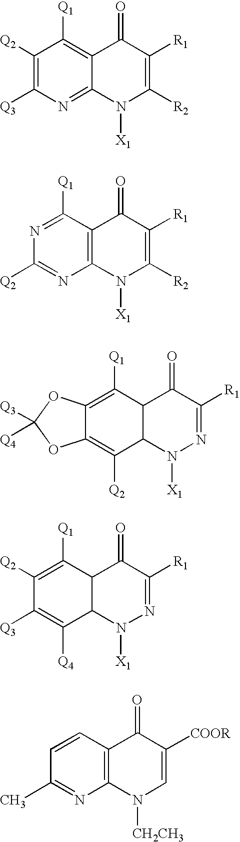

3. The cysteine protease inhibitor of claim 1, wherein said compound having the chemical structure 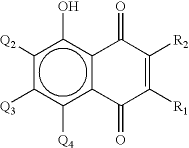
wherein one to four of the groups R₁, R₂, Q₂, Q₃, and Q₄ are hydrogen.

4. The cysteine protease inhibitor of claim 3, wherein Q₂ and Q₄ are hydrogen, hydroxy, halogen, C₁-C₃ alkyl optionally substituted with 1-3 R₁₁, and aryl ether optionally substituted with 1-5 R₁₁.

5. The cysteine protease inhibitor of claim 1, wherein said compound having the chemical structure

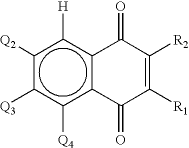

6. The cysteine protease inhibitor of claim 5, wherein Q₄ is hydrogen.

7. The cysteine protease inhibitor of claim 1, wherein said compound having the chemical structure

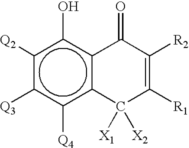

8. The cysteine protease inhibitor of claim 7, wherein Q₂ and Q₄ are hydrogen, hydroxy, halogen, C₁-C₃ alkyl optionally substituted with 1-3 R₁₁, and aryl ether optionally substituted with 1-5 R₁₁.

9. The cysteine protease inhibitor of claim 1, wherein said compound having the chemical structure

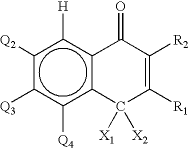

10. The cysteine protease inhibitor of claim 9, wherein Q₂ and Q₄ are hydrogen, hydroxy, halogen, C₁-C₃ alkyl optionally substituted with 1-3 R₁₁, and aryl ether optionally substituted with 1-5 R₁₁.

11. The cysteine protease inhibitor of claim 1, wherein said compound having the chemical structure

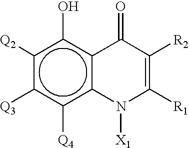

12. The cysteine protease inhibitor of claim 11, wherein Q₂ and Q₄ are hydrogen, hydroxy, halogen, C₁-C₃ alkyl optionally substituted with 1-3 R₁₁, and aryl ether optionally substituted with 1-5 R₁₁.

13. The cysteine protease inhibitor of claim 1, wherein said compound having the chemical structure

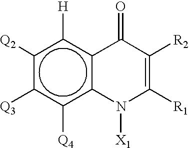

14. The cysteine protease inhibitor of claim 13, wherein Q₂ and Q₄ are hydrogen, hydroxy, halogen, C₁-C₃ alkyl optionally substituted with 1-3 R₁₁, and aryl ether optionally substituted with 1-5 R₁₁.

15. The cysteine protease inhibitor of claim 1, wherein said compound having the chemical structure

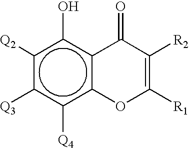

16. The cysteine protease inhibitor of claim 15, wherein Q₂ and Q₄ are hydrogen, hydroxy, halogen, C₁-C₃ alkyl optionally substituted with 1-3 R₁₁, and aryl ether optionally substituted with 1-5 R₁₁.

17. The cysteine protease rhhibitor of claim 1, wherein said compound having the chemical structure

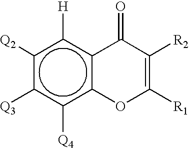

18. The cysteine protease inhibitor of claim 17, wherein Q₂ and Q₄ are hydrogen, hydroxy, halogen, C₁-C₃ alkyl optionally substituted with 1-3 R₁₁, and aryl ether optionally substituted with 1-5 R₁₁.

19. The cysteine protease inhibitor of claim 1, wherein said compound having the chemical structure

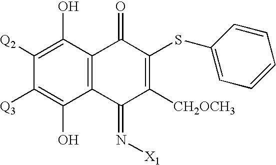

20. The cysteine protease inhibitor of claim 1, wherein said compound having the chemical structure

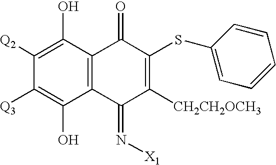

21. The cysteine protease inhibitor of claim 1, wherein said compound having the chemical structure

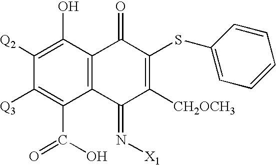

22. The cysteine protease inhibitor of claim 1, wherein said compound having the chemical structure

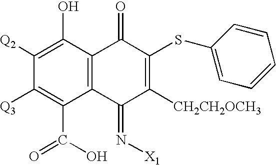

23. The cysteine protease inhibitor of claim 1, wherein said compound having the chemical structure

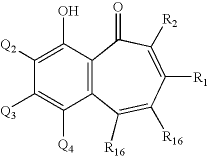

24. The cysteine protease inhibitor of claim 1, wherein said compound having the chemical structure

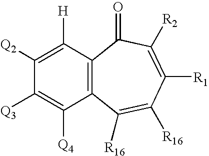

25. The cysteine protease inhibitor of claim 1, wherein said compound having the chemical structure

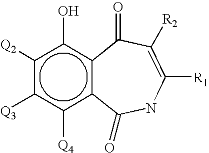

26. The cysteine protease inhibitor of claim 1, wherein said compound having the chemical structure

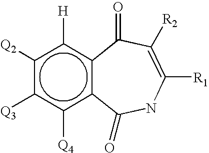

27. The cysteine protease inhibitor of claim 1, wherein said compound having the chemical structure

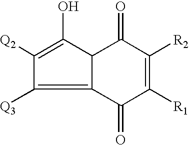

28. The cysteine protease inhibitor of claim 1, wherein said compound having the chemical structure

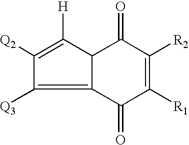

29. The cysteine protease inhibitor of claim 1, wherein said compound having the chemical structure

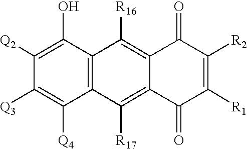

30. The cysteine protease inhibitor of claim 1, wherein said compound having the chemical structure

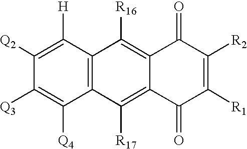

31. The cysteine protease inhibitor of claim 1, wherein said compound having the chemical structure

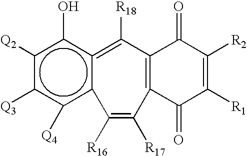

32. The cysteine protease inhibitor of claim 1, wherein said compound having the chemical structure

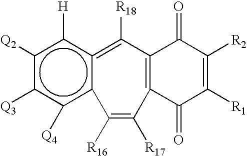

33. The cysteine protease mihibitor of claim 1, wherein said compound having the chemical structure

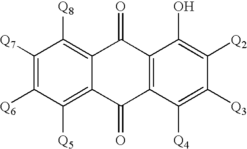

34. The cysteine protease inhibitor of claim 1, wherein said compound having the chemical structure

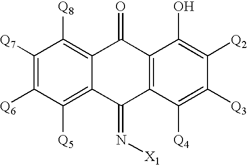

35. The cysteine protease inhibitor of claim 1, wherein said compound having the chemical structure

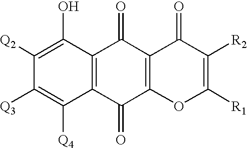

36. The cysteine protease inhibitor of claim 1, wherein said compound having the chemical structure

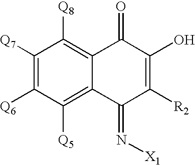

37. The cysteine protease inhibitor of claim 1, wherein said compound having the chemical structure

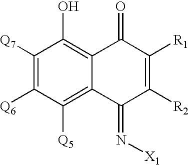

38. The cysteine protease inhibitor of claim 1, wherein said compound having the chemical structure 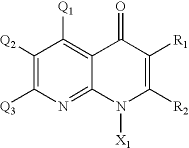
wherein X₁ is —CH₂CH₂CONH₂.

39. The chemical structure of claim 1, wherein said compound having the chemical structure 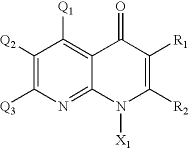
Q₁, Q₂, R₂ is H; Q₃ is -CH₃; R₁ is —COOR wherein R is CH₂O-t-butyl; and X₁ is —CH₂CH₃.

40. The chemical structure of claim 1, wherein Q₁, Q₂, R₂ is H; Q₃ is —CH₃; R₁ is —COOR wherein R is CH₂CH(CH₃)-t-butyl; and X₁ is -CH₂CH₃.

41. The cysteine protease inhibitor of claim 1, 2, 3, 4, 5, 6, ,7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40, wherein said cysteine protease inhibitor is useful for reducing apoptosis.

42. The cysteine protease inhibitor of claim 41, wherein said cysteine protease is a caspase.

43. The cysteine protease inhibitor of claim 42, wherein said cysteine protease is a caspase-3.

44. The cysteine protease inhibitor of claim 1, 2, 3, 4, 5, 6, ,7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40, wherein said cysteine protease inhibitor is useful for reducing the enzymatic activity of a 3C protease.

45. The cysteine protease inhibitor of claim 1, 2, 3, 4, 5, 6,, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43 or 44, wherein said cysteine protease inhibitor is used in a pharmaceutical preparation administered for treatment of a disease selected from the group consisting of viral diseases, neurodegenerative diseases such as Alzheimer's, Parkinson's and Huntington's diseases; spinal muscular atrophy; multiple sclerosis; immune-based diseases such as immunodeficiency, hypersensitivity, and autoimmune disorders; ischemic cardiovascular and neurological diseases or injury such as stroke, myocardial infarction, spinal cord injury or transplant organ damage; inflammatory diseases such as arthritis, cholangitis, colitis, encephalitis, endocerolitis, hepatitis, pancreatitis and reperfiusion injury; diabetes; and alopecia.

46. The cysteine protease inhibitor of claim 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43 or 44, wherein said cysteine protease inhibitor is used as an antiviral agent.

47. The cysteine protease inhibitor of claim 46, wherein said cysteine protease inhibitor is administered to nasal mucosa.

48. A method for inhibiting a cysteine protease or cysteine protease-like protein comprising contacting said cysteine protease or cysteine protease-like protein with an effective amount of a cysteine protease inhibitor according to claim 1, 2, 3, 4, 5, 6,, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40.

49. The method of claim 48, wherein said cysteine protease is a caspase.

50. The method of claim 49, wherein said cysteine protease is a caspase-3.

51. The method of claim 48, wherein said cysteine protease is a 3C-protease.

52. A method for inhibiting a cysteine protease or cysteine protease-like protein in a cell comprising contacting said cysteine protease or cysteine protease-like protein with an effective amount of a cysteine protease inhibitor according to claim 1, 2, 3, 4, 5, 6,, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40.

53. The method of claim 52, wherein said cysteine protease is a caspase.

54. The method of claim 53, wherein said cysteine protease is a caspase-3.

55. The method of claim 52, wherein said cysteine protease is a 3C-protease.

56. A method of treating a patient having a disease or disorder modulated by a cysteine protease comprising administering to said patient in need of such treatment an effective amount of a cysteine protease inhibitor according to claim 1, 2, 3, 4, 5, 6,, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40.

57. The method of claim 56, wherein said cysteine protease is a caspase.

58. The method of claim 57, wherein said cysteine protease is a caspase-3.

59. The method of claim 56, wherein said cysteine protease is a 3C-protease.

60. A cysteine protease inhibitor having a backbone structure selected from the group consisting of 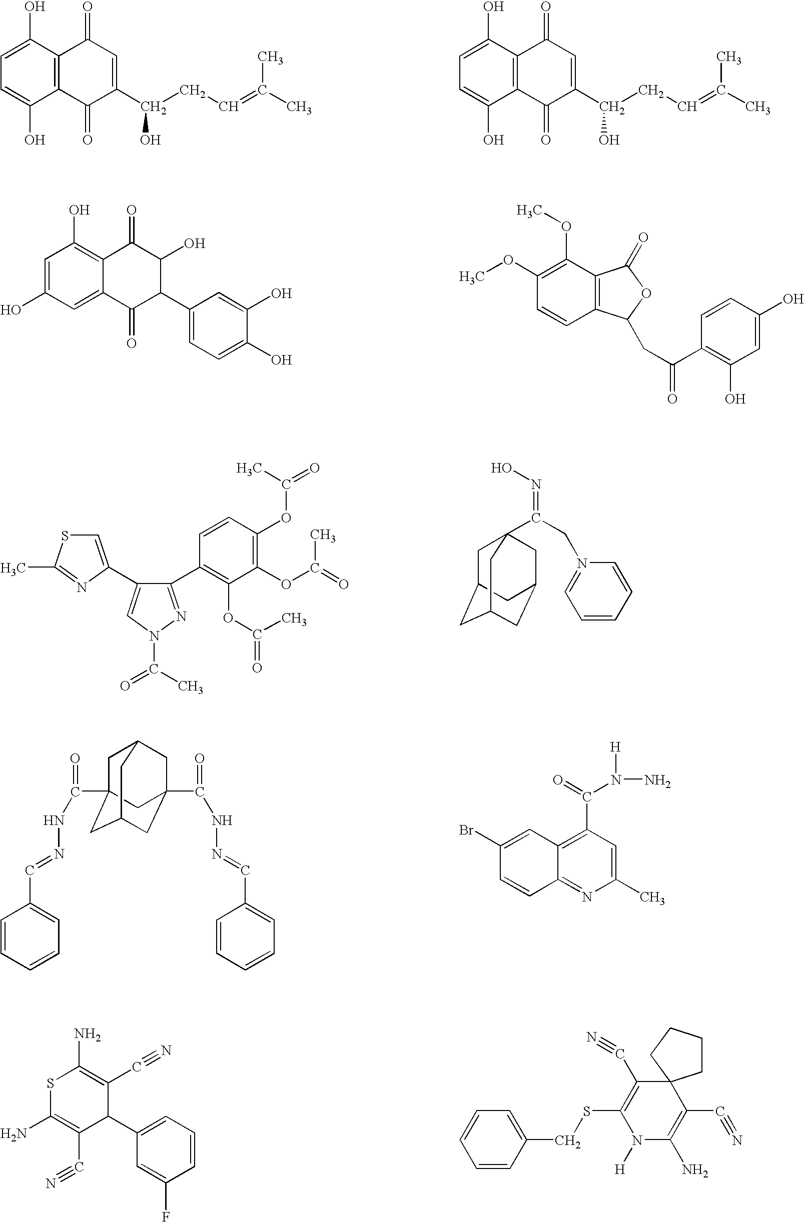 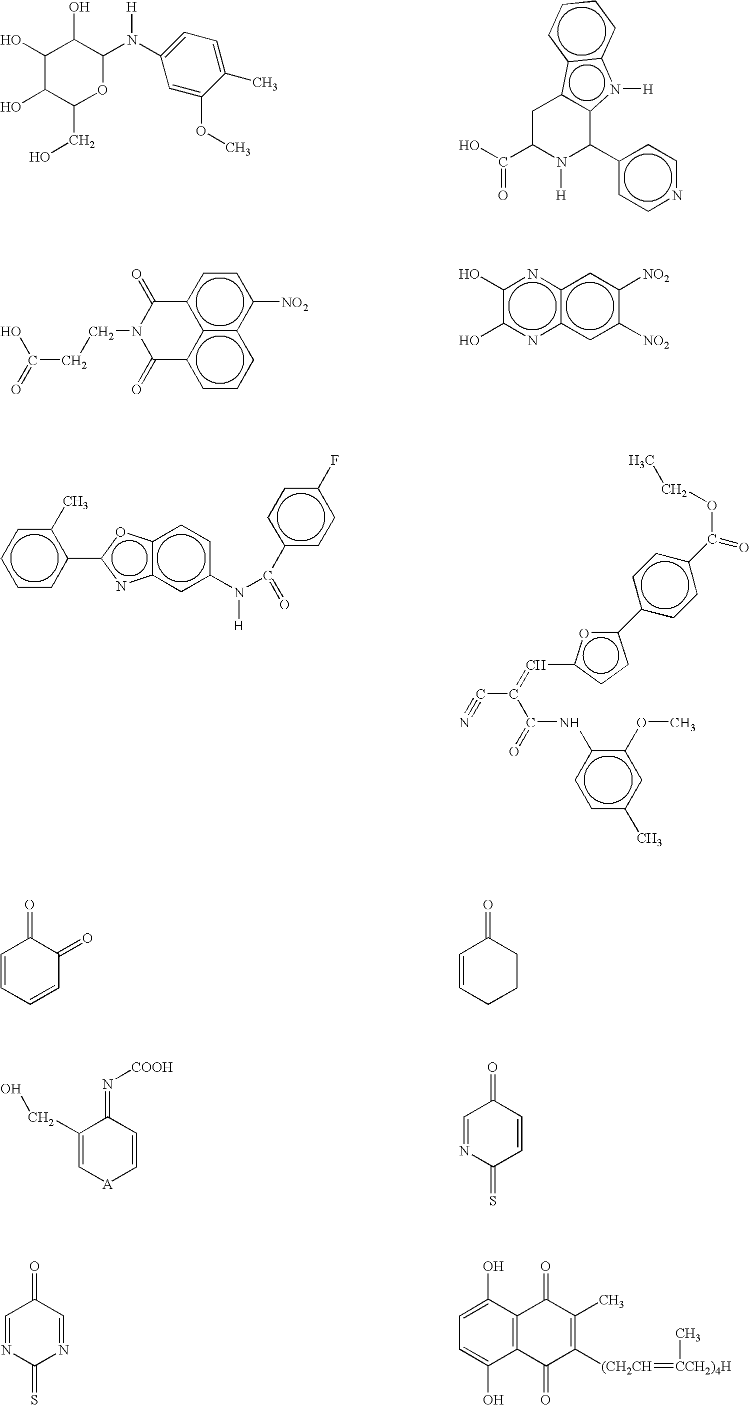 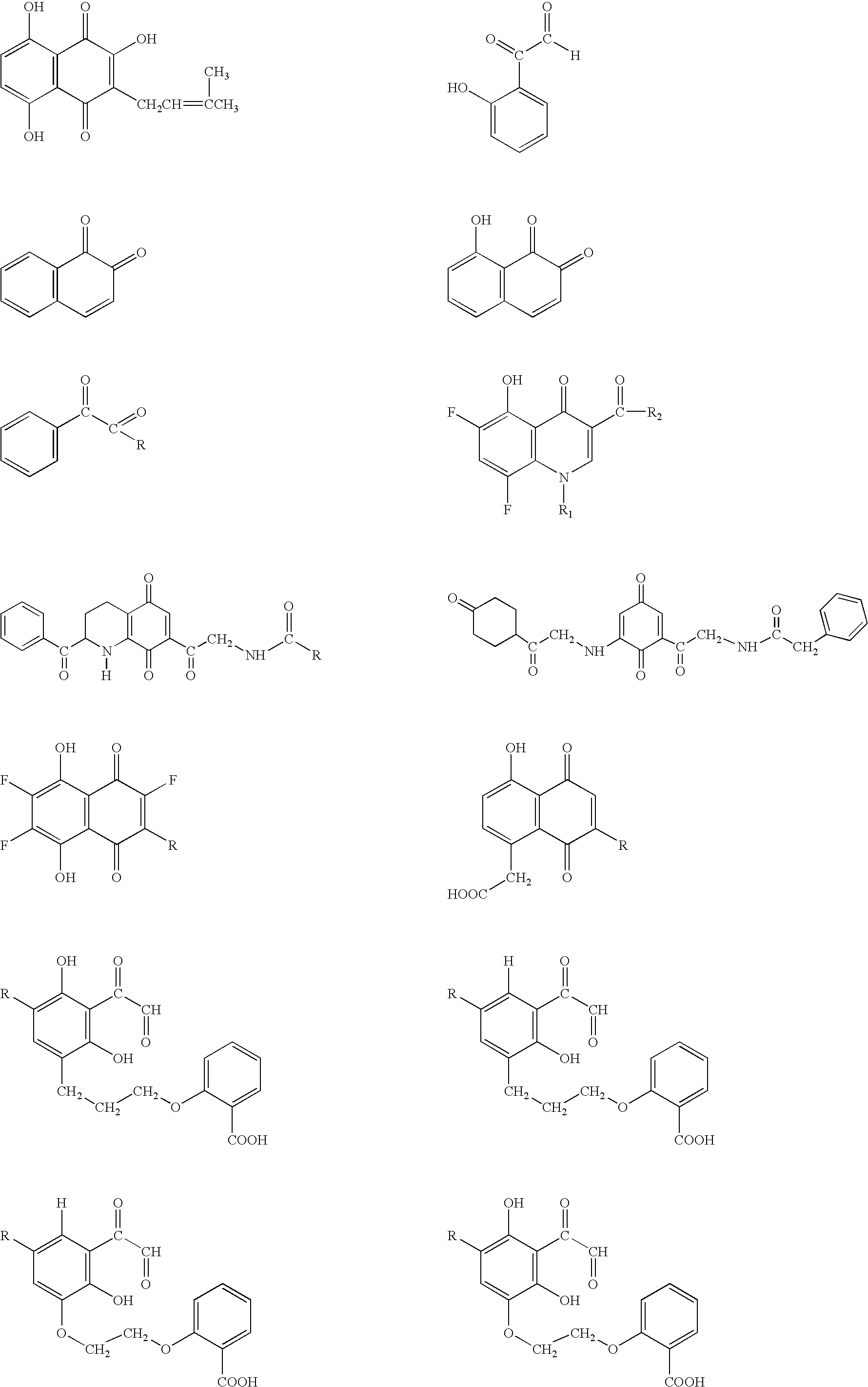 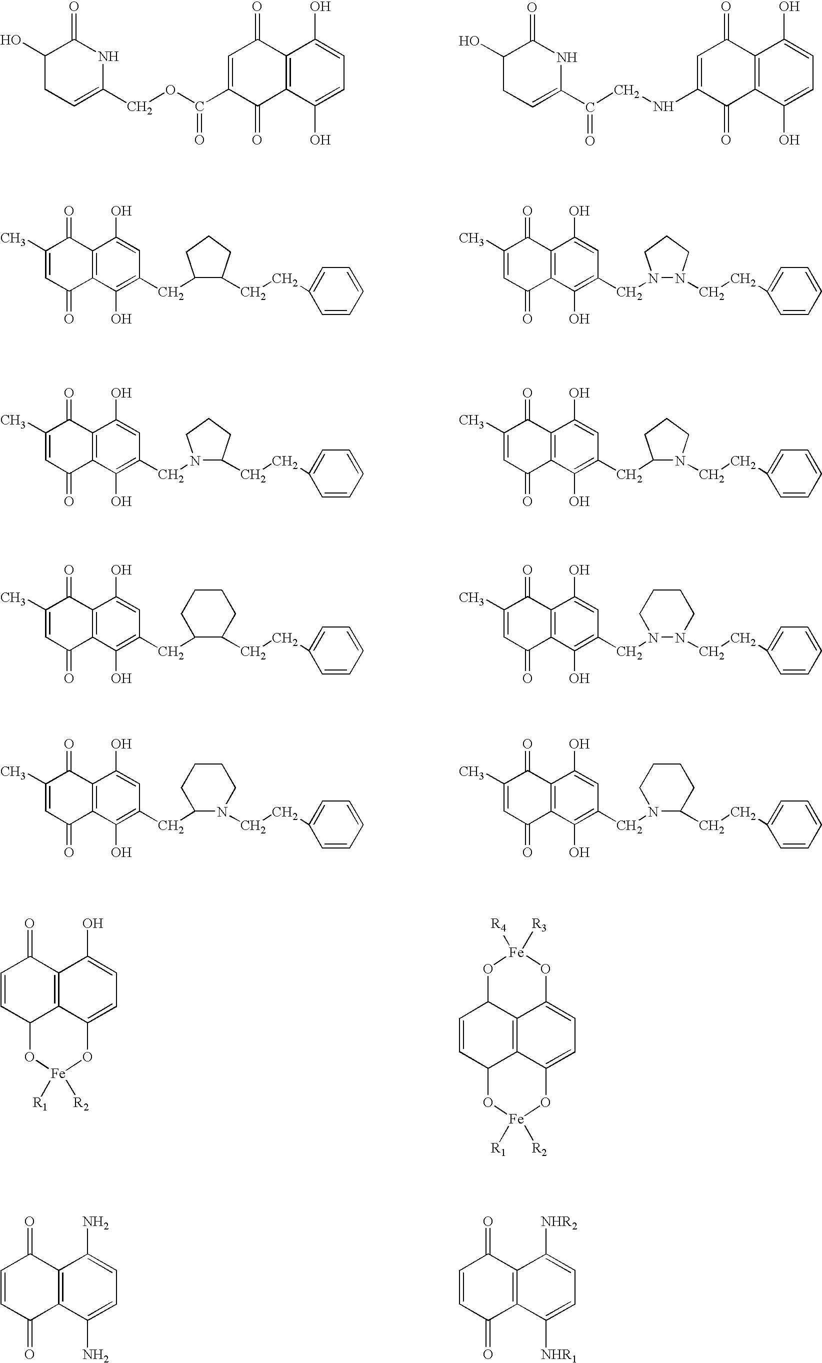 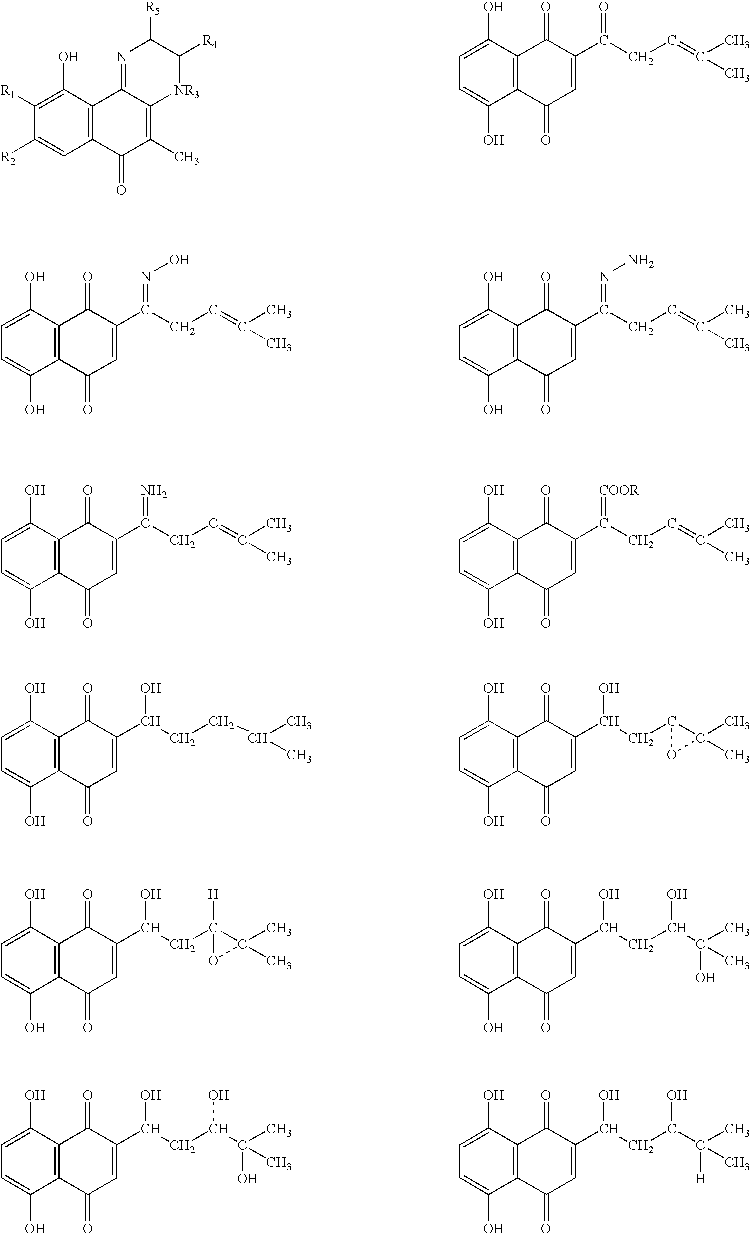 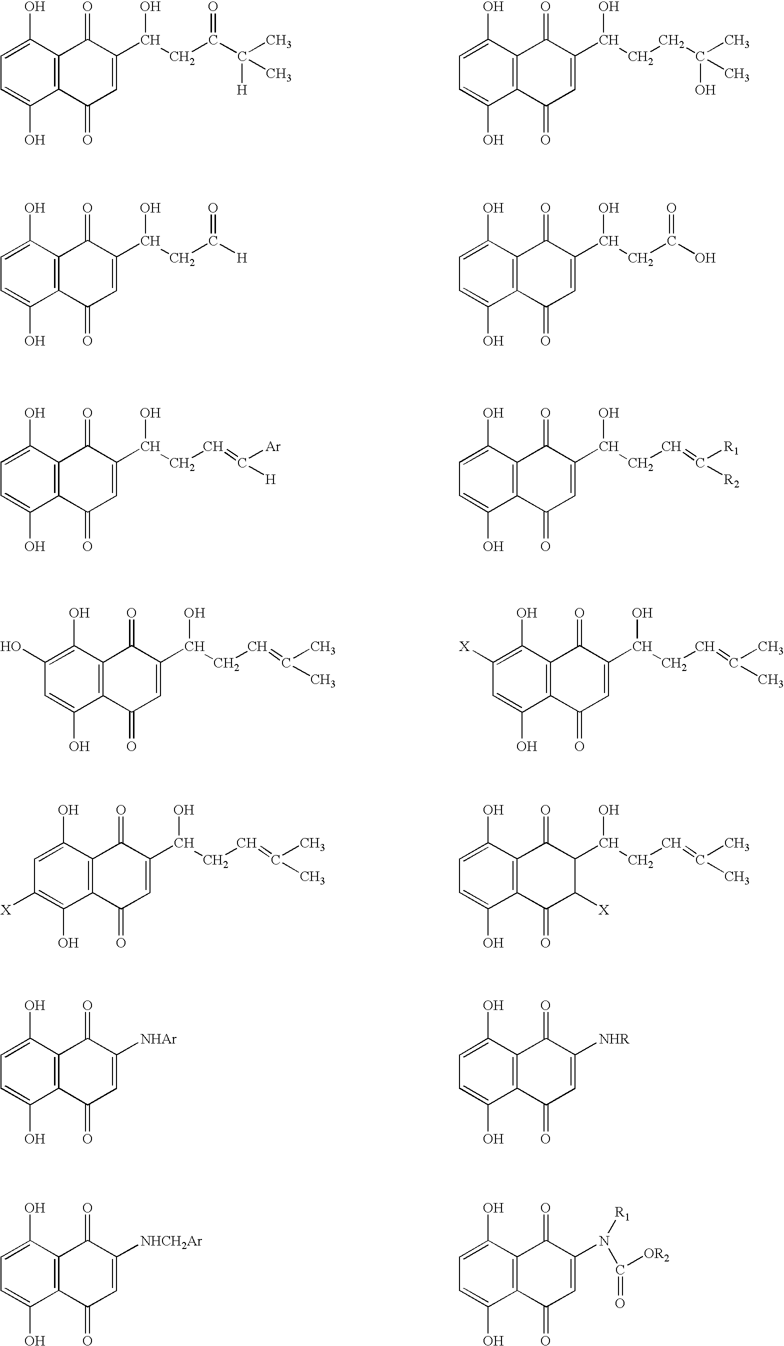 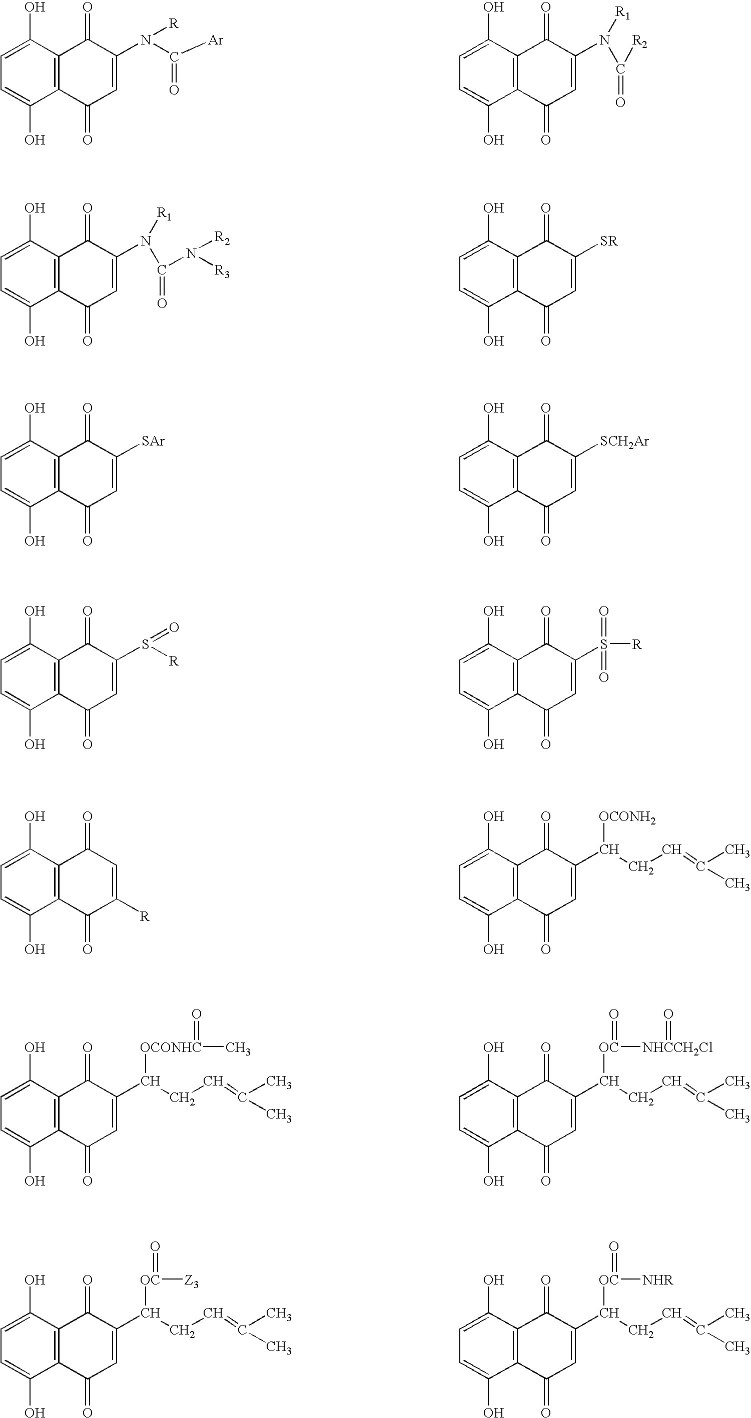 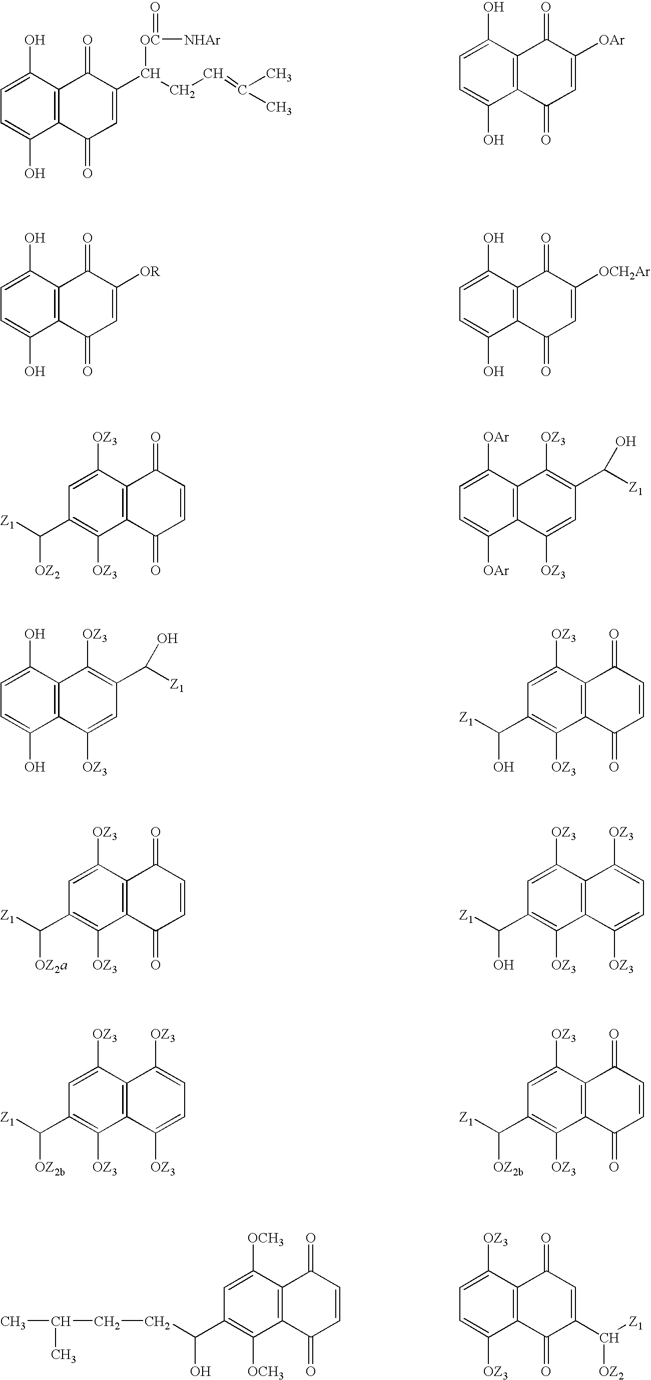 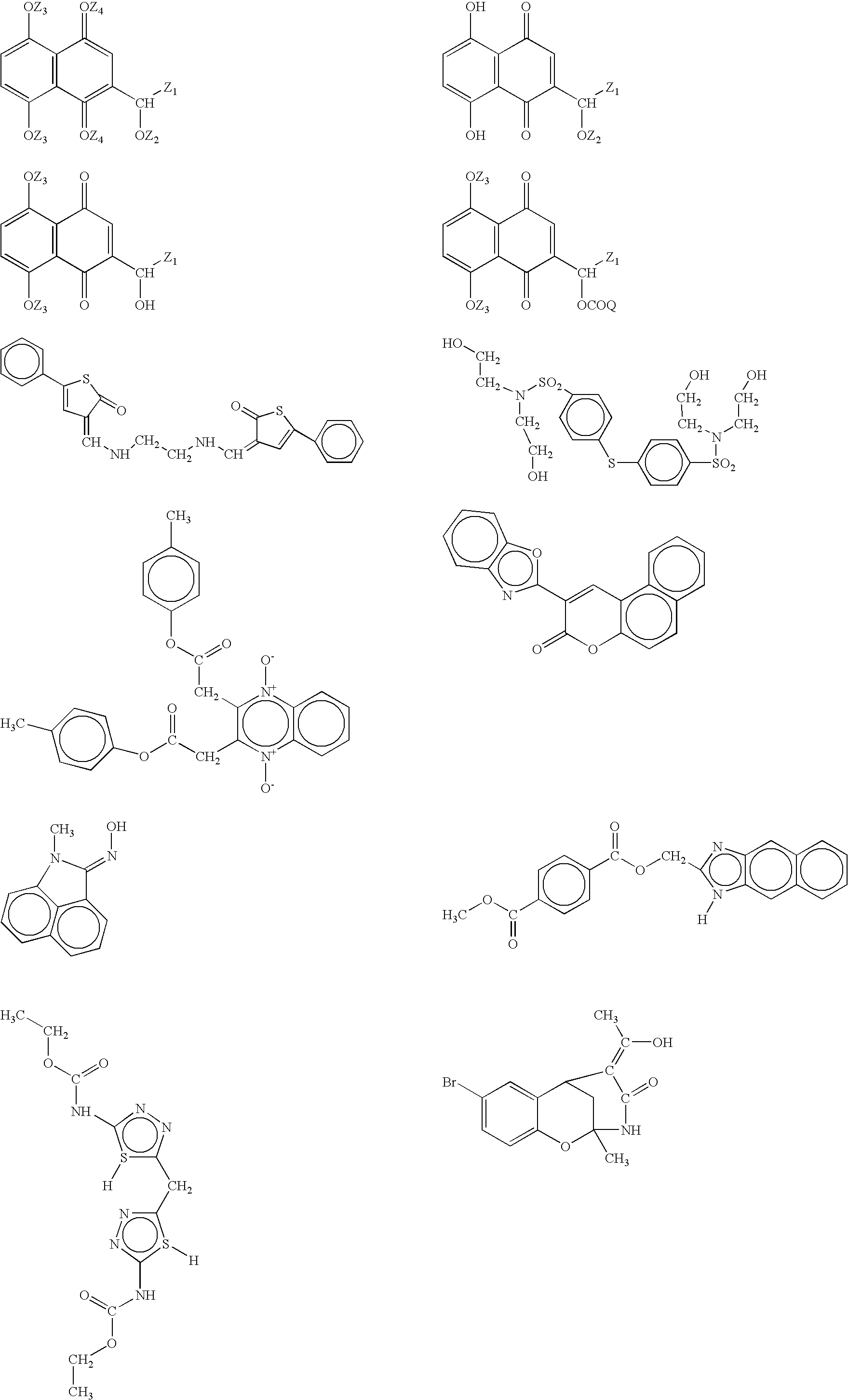 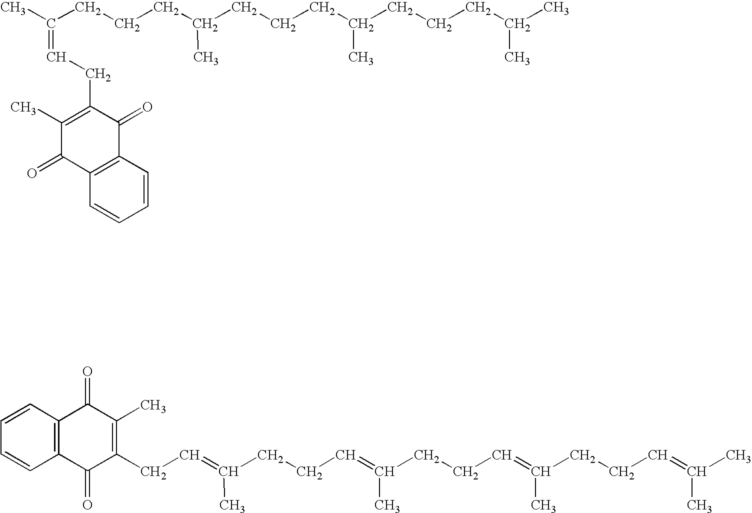
wherein A is one of the following 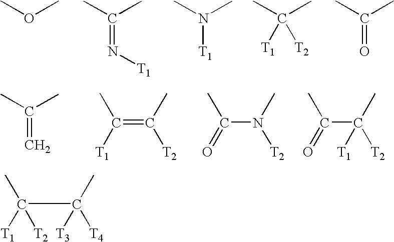
wherein T₁, T₂, T₃, T₄ are independently hydrogen, hydroxyl, halogen, methoxy, OCH₂COOH, OCH₂CONH₂, SO₂NH₂, NHSO₂NH₂, NH—Q₁, CH₂—Q1, O—Q₁, S—Q₁, C₁-C₆ alkyl with or without substitution, C₁-C₆ alkyl ether C₁-C₆ alkyl, phenyl optionally substituted with Q₁, C₃-C₁₀ cycloalkyl or bicycloalkyl optionally substituted with Q₁, C₁-C₃ alkyloxy, —NH—CO—NH₂, —NH-(3,5-dinitro-phenyl), —NH-(2,4-dinitro-phenyl) or BCl₃;
wherein R, R₁, R₂, R₃, and R₄, being the same or different, can be any organic moiety, including substituted or unsubstituted alkyl, peptide or peptide mimetic, that would fit the active site of a target cysteine protease such as caspase, e.g., caspase-3, caspase-7, caspase-8, and caspase-9;
wherein X is a halogen;
wherein Ar is a substituted or unsubstituted aryl;
wherein Z₁ is a saturated or unsaturated alkyl with or without substitution or alkenyl with or without substitution; Z₂ is hydrogen, saturated or unsaturated alkyl with or without substitution or acyl with or without substitution or a group —C(O)Q wherein Q is alkyl, alkenyl, aryl, aralkyl or aralkenyl with or without substitution; Z₂ₐ is acyl with or without substitution; Z_{2b} is a saturated or unsaturated alkyl with or without substitution; Z₃ is hydrogen or a saturated or unsaturated alkyl with or without substitution; and Z₄ is saturated or unsaturated alkyl with or without substitution; and
wherein any —OH group at the side chain C(2′) position can be alpha and beta stereochemistry.

61. The cysteine protease inhibitor of claim 60, wherein said cysteine protease inhibitor is useful for reducing apoptosis.

62. The cysteine protease inhibitor of claim 60, wherein said cysteine protease is a caspase.

63. The cysteine protease inhibitor of claim 62, wherein said cysteine protease is a caspase-3.

64. The cysteine protease inhibitor of claim 60, wherein said cysteine protease inhibitor is useful for reducing the enzymatic activity of a 3C protease.

65. The cysteine protease inhibitor of claim 60, 61, 62, 63 or 64, wherein said cysteine protease inhibitor is used in a pharmaceutical preparation administered for treatment of a disease selected from the group consisting of viral diseases, neurodegenerative diseases such as Alzheimer's, Parkinson's and Huntington's diseases; spinal muscular atrophy; multiple sclerosis; immune-based diseases such as immunodeficiency, hypersensitivity, and autoimmune disorders; ischemic cardiovascular and neurological diseases or injury such as stroke, myocardial infarction, spinal cord injury or transplant organ damage; inflammatory diseases such as arthritis, cholangitis, colitis, encephalitis, endocerolitis, hepatitis, pancreatitis and reperfusion injury; diabetes; and alopecia.

66. The cysteine protease inhibitor of claim 65, wherein said pharmaceutical preparation further comprises at least one compound selected from the group consisting of DTT or a derivative, HSCH₂CH₂OHCH₂OHCH₂SH, GSH (glutathione), HOOCCH(NH₂)CH₂CH₂CONHCH(CH₂SH)CONHCH₂COOH, mycothiol (MT), any other sulfur-reducing agent, any adduct of naphthoquinone derivative and DTT or GSH or MT or any adduct with a different oxidation state (e.g., NO^{*-} or NOH^{*-}), and

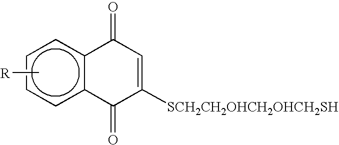

67. A method for inhibiting a cysteine protease or cysteine protease-like protein comprising contacting said cysteine protease or cysteine protease-like protein with an effective amount of a cysteine protease inhibitor according to claim 60, 61, 62, 63 or 64.

68. The method of claim 67, wherein said cysteine protease is a caspase.

69. The method of claim 68, wherein said cysteine protease is a caspase-3.

70. The method of claim 67, wherein said cysteine protease is a 3C-protease.

71. A method for inhibiting a cysteine protease or cysteine protease-like protein in a cell comprising contacting said cysteine protease or cysteine protease-like protein with an effective amount of a cysteine protease inhibitor according to claim 60, 61, 62, 63 or 64.

72. The method of claim 71, wherein said cysteine protease is a caspase.

73. The method of claim 72, wherein said cysteine protease is a caspase-3.

74. The method of claim 71, wherein said cysteine protease is a 3C-protease.

75. A method of treating a patient having a disease or disorder modulated by a cysteine protease comprising administering to said patient in need of such treatment an effective amount of a cysteine protease inhibitor according to according to claim 60, 61, 62, 63 or 64.

76. The method of claim 75, wherein said cysteine protease is a caspase.

77. The method of claim 76, wherein said cysteine protease is a caspase-3.

78. The method of claim 75, wherein said cysteine protease is a 3C-protease.

79. A method for the treatment of diseases or disorders affected by cysteine protease activity comprising administration of at least one of the group consisting of alkannin, alkannin naphthoquinone derivative, shikonin, and shikonin naphthoquinone derivative.

80. The method of claim 79, wherein said cysteine protease is a caspase.

81. The method of claim 80, wherein said cysteine protease is a caspase-3.

82. The method of claim 79, wherein said cysteine protease is a 3C-protease.

83. A method for the treatment of excessive apoptosis affected by cysteine protease activity in a cell comprising administration of at least one of the group consisting of alkannin, alkannin naphthoquinone derivative, shikonin, and shikonin naphthoquinone derivative.

84. The method of claim 83, wherein said cysteine protease is a caspase.

85. The method of claim 84, wherein said cysteine protease is a caspase-3.

86. The method of claim 83, wherein said cysteine protease is a 3C-protease.

87. A method for the treatment of viral diseases comprising administration of a formulation having at least one compound of the group consisting of nalidixic acid and derivatives.

88. The method of claim 87, wherein said compound is nalidixic acid.

89. The method of claim 88, wherein said compound is effective for picomaviruses, rhinoviruses, hepatitis viruses, immunodeficienty viruses, and influenza viruses.

90. The method of claim 87, wherein said compound is a nalidixic acid derivative selected from the group consisting of C₁-C₆ alkyl, unsubstituted or substituted with an oxymethyl group, phenyl, and substituted aryl.

91. The method of claim 90, wherein said compound is effective for picornaviruses, rhinoviruses, hepatitis viruses, immunodeficienty viruses, and influenza viruses.

92. The method of claim 88 and 90, wherein said formulation is administered to nasal mucosa.
